(19) United States
(12) Patent Application Publication
(10) Pub. No.: US 2015/0004161 A1
(43) Pub. Date: Jan. 1, 2015

(54) Fc Coupled Compositions and Methods of Their Use

(71) Applicant: University of Maryland, College Park, MD (US)

(72) Inventor: Xiaoping Zhu, Clarksville, MD (US)

(21) Appl. No.: 14/321,412

(22) Filed: Jul. 1, 2014

Related U.S. Application Data
(60) Provisional application No. 61/841,755, filed on Jul. 1, 2013.

(51) Int. Cl. C07K 14/705; A61K 39/00

(52) U.S. Cl. CPC C07K 14/70521; A61K 39/0005; A61K 2039/57; C07K 2319/30; A61K 2039/54
USPC 424/134.1; 530/387.3

(57) ABSTRACT

Disclosed are compositions comprising an Fc portion of IgE coupled to an agent. For example, disclosed are compositions comprising an Fc portion of IgE coupled to an antigen or immunotherapeutic. These compositions can be used as a vaccine or an immunotherapeutic. Thus, these compositions can modulate the immune system by both increasing and decreasing the immune response. The Fc portion of IgE can bind to CD23 and transport the antigen or immunotherapeutic across airway epithelial cells. Disclosed are methods of treating airway inflammation with compositions comprising an Fc portion of IgE coupled to an antigen or immunotherapeutic.

### CROSS-REFERENCE TO RELATED APPLICATIONS

This application claims benefit of U.S. Provisional Application No. 61/841,755, filed Jul. 1, 2013. Application No. 61/841,755, filed Jul. 1, 2013, is hereby incorporated herein by reference in its entirety.

### STATEMENT REGARDING FEDERALLY SPONSORED RESEARCH

This invention was made with government support under R21AI101752 awarded by National Health of Institutes (NIH). The government has certain rights in the invention.

### FIELD OF THE INVENTION

The disclosed invention is generally in the field of immune modulation and specifically in the area of respiratory immune modulation.

### BACKGROUND

Respiratory diseases, disorders and infections are extremely common. In particular, respiratory diseases that cause airway inflammation can be highly dangerous due to the closing of the airway and decreased ability to breath. Airway inflammation is characterized by bronchial hyper-responsiveness and intermittent airway obstruction with an underlying Th2 cell-biased inflammatory response. These types of diseases are currently treated with bronchodilators or anti-inflammatory drugs such as corticosteroids, leukotriene modifiers, and anti-IgE therapy. However, the current treatments are not curative and some patients do not respond well to intense anti-inflammatory therapies. Additionally, the use of long-term steroids can result in many undesired side effects. Thus, it is important to develop more effective therapies for airway inflammation.

### BRIEF SUMMARY

Disclosed are compositions comprising an Fc portion of IgE coupled to an antigen. The antigen can be an allergen, immunomodulating proteins, or can be derived from a virus, bacteria, parasite or fungus.

Antigens can be allergens. Allergens can be an antigen derived from pollens, dust, mites, molds, spores, dander, insects or foods. The allergen can be urushiols (pentadecylcatechol or heptadecylcatechol) or sesquiterpenoid lactones.

Antigens can be a tumor antigen. Antigens can be a cell surface, cytoplasmic, nuclear, or mitochondrial antigen.

Disclosed are compositions comprising an Fc portion of IgE coupled to an immunotherapeutic agent. Immunotherapeutic agents can be Cytotoxic T lymphocyte antigen 4 (CTLA4), Soluble IL-4 receptor, Soluble IL-13 receptor, IL-5R, Thymic stromal derived lymphopoietin receptor (TSLPR), IL10, IL-9 receptors, IL-17 receptors, IL-25 receptors, IL-31 receptors, IL-33 receptors, transforming growth factor beta (TGFbeta), Histamine receptors, Prostaglandin receptors, FcepsilonRI alpha, programmed death 1 (PD1), Flt3-ligand, leukotriene receptor, Tumor necrosis factor (TNFR), LIGHT receptor, OX40L, IL-1beta receptor, c-kit, ADAM, soluble intercellular adhesion molecule 1 (sICAM-1), soluble IL-2R, CD48, Pulmonary surfactant protein D (SPD), soluble β2-Adrenergic receptor, B7-1, B7-H1, leukocyte-associated immunoglobulin-like receptor 1 (LAIR1), lymphocyte activation gene 3 protein (LAG3), CD160, LAG3, lymphocyte activation gene 3 protein (TIGIT), type I transmembrane (or T cell) immunoglobulin and mucin. (TIM3), B and T lymphocyte attenuator (BTLA).

Disclosed are IgE Fc compositions further comprising a pharmaceutically acceptable carrier.

Disclosed are vaccines comprising the disclosed compositions comprising the Fc portion of IgE coupled to an antigen.

Disclosed are methods of treating airway inflammation comprising administering to a subject an effective amount of the disclosed compositions comprising the Fc portion of IgE coupled to an antigen or an immunotherapeutic. Airway inflammation can be from asthma or allergies.

Disclosed are methods of interfering with Th2 cell activation comprising administering to a subject an effective amount of the disclosed compositions comprising the Fc portion of IgE coupled to an antigen or immunotherapeutic. The immunotherapeutic agent can be CTLA4. The composition can block signaling through a CD28 molecule on the T cell.

Disclosed are methods of blocking mast cell degranulation comprising administering to a subject an effective amount of the disclosed compositions comprising the Fc portion of IgE coupled to an antigen or immunotherapeutic agent. The composition can bind to Fc receptors on the surface of the mast cell and prevent IgE from binding to the Fc receptors.

Disclosed are methods of decreasing proinflammatory cytokines in the bronchoalveolar lavage fluid (BALF) comprising administering to a subject an effective amount of a composition comprising the Fc portion of IgE coupled to immunotherapeutic agent.

Disclosed are methods of reducing the numbers of hematopoietic cells in the BALF comprising administering to a subject an effective amount of a composition comprising the Fc portion of IgE coupled to immunotherapeutic agent. The hematopoietic cells can be eosinophils or macrophages.

The disclosed methods can have administration of the disclosed compositions wherein the administration can be intranasal.

Disclosed are methods of modulating the immune system comprising administering to a subject an effect amount of the composition comprising the Fc portion of IgE coupled to an antigen or immunotherapeutic agent. The compositions administered in the methods can further comprise an adjuvant. The compositions can elicit an immune response to the antigen. The modulating of the immune system can comprise inducing an immune response. The modulating of the immune system can comprise reducing an immune response.

Disclosed are methods of delivering an antigen across airway epithelial cells comprising administering to a subject an effect amount of a composition comprising the Fc portion of IgE coupled to an antigen.

Disclosed are methods of delivering an immunotherapeutic across airway epithelial cells comprising administering to a subject an effect amount of a composition comprising the Fc portion of IgE coupled to an immunotherapeutic agent.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic description of CD23 mediated immunotherapy. CD23 expressed in the airway epithelium binds to IgE-Fc fusion protein in the airway lumen and transports immune therapeutic proteins across the epithelial barrier for immunotherapeutic purpose.

FIGS. 2A, 2B and 2C show the construction, purification and expression of mouse CTLA4-IgE Fc fusion protein. (A) Construction of mouse CTLA4-IgE Fc plasmid. The mRNA for mouse CTLA4 (lane 2) and IgE Fc (lane 3) fused together using PCR based assembly (lane 4) and double enzyme digested pcDNA3 plasmid containing mouse CTLA4-IgE Fc (lane 5). (B) Purification of fusion protein. The fusion protein purified using either anti-mouse IgE B1E3 or anti-mouse CTLA4 UC10-4F10-11 conjugated Sepharose 4B beads were electrophoresed and separated on 12% SDS-PAGE gel under reducing condition (lane 1) or non-reducing condition (lane 2). The separated proteins were transferred onto nitrocellulose membrane, blocked and blotted with goat anti-mouse IgE antibody. The blots were washed and further incubated with HRP-conjugated bovine anti-goat IgG antibody and the protein bands were visualized by enhanced chemiluminance (ECL) method. The arrow indicates the location of mouse IgE Fc. (C) Detection of fusion protein using ELISA. The purified fusion protein was detected by ELISA method. Normal CHO cell culture supernatant was used as control. Purified fusion protein gives stronger OD450 value and gradually decreased upon serial dilution of fusion protein. Control CHO cell culture supernatant gives background value.

FIGS. 3A, 3B, and 3C show the characterization of mouse CTLA4-IgE Fc fusion protein. (A) Mouse CTLA4-IgE Fc fusion proteins bind to mouse B7-1 and B7-2 expressed on CHO cells. The binding capability was analyzed by flow cytometry. Cells were stained with anti-mouse IgE FITC antibody. Results are expressed as histograms of fluorescence intensity (log scale). Values in each rectangle correspond to the proportion of cells binds to fusion protein. Mouse IgE was used as isotype control. Binding of fusion protein to normal CHO cells represents the background staining Fusion protein, mouse CTLA4IgE Fc, binds strongly to CHO cells expressing mB71/mB72 when compared to control CHO cells. (B) Mouse CTLA4-IgE Fc fusion protein bind to rat mast RBL-2H3 cells. The binding affinity of mouse CTLA4-IgE Fc to Fc RI expressed on RBL-2H3 cells was analyzed by flow cytometry. Cells were stained with anti-mouse IgE FITC antibody. Results are expressed as histograms of fluorescence intensity (log scale). Values in rectangle correspond to the proportion of cells binds to fusion protein or positive control mouse IgE. Negative control was performed without the addition of any protein. Fusion protein, mouse CTLA4IgE Fc, binds strongly to mast RBL-2H3 cells. (C) Fusion protein, mouse CTLA4-IgE Fc, binds to spleen cells. The binding affinity of mouse CTLA4-IgE Fc to CD23 expressed on mouse B cells was analyzed by flow cytometry. Total spleen cells were obtained from either wild-type or CD23 knockout (KO) mice. Cells were stained with anti-mouse IgE FITC and B220 APCCy7 antibody. Results are expressed as histograms of fluorescence intensity (log scale). Values in rectangle on the left side correspond to the proportion of total spleen cells that bind to fusion protein obtained from wild-type or CD23KO mice. Values in the quadrant on the right side corresponds to the B220 positive B cells obtained from wild-type or CD23KO mice that bind to fusion protein. Negative control was performed without the addition of any protein. Wild-type mice obtained total spleen cells and B220 positive B cells binds to the fusion protein significantly when compared to the CD23KO mice.

FIG. 4 shows that the fusion protein, CTLA4-IgE Fc, blocks the degranulation of RBL-2H3 cells. RBL-2H3 cells (2×10⁴/well) were grown overnight in 48 well plate. Cells were sensitized with biotinylated IgE in the presence or absence of fusion protein and cross-linked for inducing degranulation by streptavidin. Cell degranulation was measured by f3-Hexosaminidase release in the supernatant and was expressed in the percentage of the total cellular concentration. Spontaneous degranulation was performed without addition of biotinylated IgE. As a positive control, RBL-2H3 cells degranulated with biotinylated IgE and streptavidin releases β-Hexosaminidase in a time dependent manner. Addition of fusion protein at different concentrations inhibits the release of β-Hexosaminidase to the level of spontaneous negative control samples.

FIG. 5 shows that CTLA4-Fc fusion proteins significantly reduced IL-4 and IFN-g cytokine release in the spleen cells that were isolated from ovalbumin sensitized mice. An in-vitro antigen recall assay is shown. Total spleen cells (2×10⁶/well) isolated from the mice sensitized with ovalbumin were treated either with mock or mouse CTLA4-IgE Fc fusion protein (1 μg/ml) and then stimulated with ovalbumin (50 μg/ml). IL-4 and IFN-γ secreted from spleen cells were measured by ELISA method. Both IL-4 and IFN-γ secretion from the fusion protein mouse CTLA4-IgE Fc treated samples were significantly reduced when compared to mock-treated samples.

FIGS. 6A, 6B, 6C and 6D show that CD23 transports mouse CTLA4-IgE Fc fusion proteins across mouse tracheal epithelial cell monolayer. (A) Immunofluorecence staining of the tight junction protein. Mouse tracheal epithelial cells grown on Transwell insert was fixed and immunostained with anti-E-cadherin antibody (green) and the nucleus with DAPI. (B)-(D) Mouse tracheal epithelial cells transports mouse CTLA4-IgE Fc fusion protein and mouse IgE. Mouse tracheal epithelial cells isolated from wild type or CD23 KO mice were grown to allow polarization on transwell filters. Mouse CTLA4-IgE Fc fusion protein (B) was added to apical chamber. As a positive control, mouse IgE was added either to apical (D) or basolateral (C) chambers and incubated at 37° C. for 2 hrs. The medium from opposite chamber was collected and mouse CTLA4-IgE Fc or IgE concentration was measured by ELISA. Mouse tracheal epithelial cells obtained from the wild-type mice transport mouse CTLA4-IgE Fc fusion proteins or IgE across the polarized epithelial monolayer significantly when compared to the tracheal epithelial cells freshly isolated CD23KO mice.

FIG. 7 shows a schematic description of a mouse allergy model. Wild-type mice were intraperitoneally (i.p.) sensitized with 100 μg OVA dissolved in PBS plus 4 mg Alum at day 0 and subsequently with i.p. injection of 100 μg OVA dissolved in PBS at day 7 and 14, respectively. On day 20 and 21 the mice were treated either with mouse CTLA4-IgE Fc fusion protein (5 μg dissolved in PBS) or with PBS for the control mice. On day 21, 22 and 23 the mice were challenged with nebulized 1% OVA for 30 min. Mice were sacrificed 24 h after last aerosol challenge and analyzed for allergy development.

FIGS. 8A, 8B, and 8C show the therapeutic effect of the CTLA4-Fc in ova-based asthma model. Immunotherapy with mouse CTLA4-IgE Fc proteins reduces type 2 T cell (Th2) mediated inflammation. (A)-(C) Mice were sensitized with OVA antigen. Before challenge, OVA-sensitized eild-type mice intranasally treated either with 5 μg mouse CTLA4-IgE Fc or with PBS. Then the treated mice were challenged with nebulized 1% OVA in PBS for 30 min. Bronchoalveolar lavage (BAL) fluid and serum were collected 24 hours after last OVA airway challenge. Concentrations of IL-4 (A) and OVA-specific IgE antibody (B) in either BAL or sera were measured by ELISA, respectively. OVA-specific IgE and IL-4 were significantly reduced in the BAL obtained from mouse CTLA4-IgE Fc treated mice when compared to PBS treated group of mice. The number of eosinophil was counted by flow cytometric analysis of CD45+ CD11bhi/int CD11c- Siglec-F+ cells in BAL fluid (C). Eosinophil counts were significantly reduced in mouse CTLA4-IgE Fc treated mice. *P<0.05.

FIG. 9 shows the effect of mouse CTLA4-IgE Fc targeting to epithelial CD23 on airway allergic inflammation. Mice were sensitized with OVA. Before challenge, OVA-sensitized WT mice intranasally treated either with 5 g mouse CTLA4-IgE Fc or PBS. Then the mice were challenged with nebulized 1% OVA for 30 min. Mice were sacrificed 28 hours after last OVA challenge and lung tissues were fixed with formalin and embedded with paraffin. Lung sections were stained with periodic acid-Schiff staining Mouse CTLA4-IgE Fc treated mice showed a significant reduction in goblet-cell hyperplasia in the lung bronchial area (top panel) in comparison with that of the PBS treated mice (bottom panel).

FIG. 10 shows a schematic representation of membrane-bound CD23 structure. CD23 has the extracellular trimeric α-helical coiled-coil ‘stalk’, the three C-type lectin domain ‘heads’ and the C-terminal ‘tails’. N-linked glycosylation sites are near the base of the stalk. C-terminal lectin domain is the site of interaction with the CH3 domain of IgE.

FIG. 11 shows a blot performed to detect IgE heavy chain. Calu-3 cells (1×10⁵/ml) were grown on 0.4 μm pore size transwell inserts. The cells were allowed to get polarized and the transcytosis assay was performed when the TER value reached at least 450-900 Ω/cm². The human IgE (0.5 μmmol/L) was added either to apipcal or basolateral chamber and incubated at 37° C. for 2 hr. For blocking IgE transcytosis, the Calu-3 cells in inddvidual inserts were incubated at 4° C. for 2 hrs. The IgE was subsequently added and transcytosis assay was performed for additional 2 hr at 4° C. The medium from opposite chamber was collected, concentrated and blotted to detect IgE heavy chain. A: apical; B: basolateral.

FIG. 12 shows CD23 expression was detected in tracheal epithelial cells by immunofluorescence staining with B3B4 mAb. NC: negative control; DAPI: nucleus staining.

FIG. 13 shows images of IgE and CD23 staining Biotin-IgE (50 mg) was i.n. inoculated into mice for 5 minutes. IgE was colocalized with CD23 in the epithelial cells of trachea. Arrows indicate the transported biotin-IgE.

FIG. 14 is a graph showing anti-ova IgE. Biotin-IgE was detected in the serum by ELISA after biotin-IgE was i.n. administered into mice. ND: not detected.

FIG. 15 is a model of CD23 mediated transport of IgE-Fc fusion proteins across the airway mucosal barrier.

FIG. 16 shows a construction of IgE Fc-based fusion gene CTLA4-Fc expression vector in pcDNA3.

FIG. 17 shows the production of CTLA4-Fc fusion proteins from CHO cells. Medium was run in SDS-PAGE gel under reducing conditions. The proteins were detected by antibody against IgE-Fc and ECL method.

FIG. 18 shows CD23 expressions in the trachea and lung were enhanced after mice were i.p. sensitized with OVA in comparison with those of naïve mice. CD23 was detected by B3B4 specific mAb in a Western blot. N: naïve; S: sensitized.

FIG. 19 shows TSLP expression was induced in airway epithelial cells lining trachea and lung in OVA-sensitized mice. TSLP was detected by specific mAb.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

It must be noted that as used herein and in the appended claims, the singular forms “a”, “an”, and “the” include plural reference unless the context clearly dictates otherwise. Thus, for example, reference to “a composition” includes a plurality of such compositions, reference to “the NK cell” is a reference to one or more NK cells and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range [symbol] from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. If a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C—F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. FC COUPLED COMPOSITIONS

Disclosed are compositions comprising an Fc portion of IgE coupled to an agent. Agents can be antigens or immunotherapeutic agents. Thus, disclosed are compositions comprising an Fc portion of IgE coupled to an antigen and also disclosed are compositions comprising an Fc portion of IgE coupled to an immunotherapeutic.

1. IgE

IgE is a major player in airway allergic inflammation. IgE is known to bind CD23 present on many cells, including but not limited to B cells, macrophages, eosinophils, dendritic cells, platelets and epithelial cells. CD23 is also known as Fc epsilon Receptor II. CD23 can transport IgE across intestinal epithelia.

The Fc portion of IgE can be coupled or conjugated to an antigen or immunotherapeutic agent. AS used herein, the Fc portion of IgE” refers to the constant region of an IgE antibody. Fc portion of IgE can also be referred to as IgE Fc.

Wild type or mutated sequences of IgE Fc can be used as long as the resulting IgE Fc can bind CD23 and be transported across epithelial cells. Furthermore, any of the compositions or methods disclosed herein can comprise a full IgE molecule in a conjugated form in place of the IgE Fc portion. Thus, a modified IgE antibody can be used instead of the Fc fragment of the IgE antibody.

“Fc coupled compositions” refers to compositions containing an Fc portion coupled to another molecule. IgE Fc coupled compositions refer to compositions containing IgE Fc coupled to another molecule. Fc coupled compositions can also refer to whole antibodies coupled to another molecule. Thus, IgE Fc coupled compositions can refer to the whole IgE antibody coupled to another molecule.

As used herein, the term “coupled” means that the specified moieties are either directly covalently bonded to one another, or indirectly covalently joined to one another through an intervening moiety or moieties, such as a bridge, spacer, or linkage moiety or moieties, or they are non-covalently coupled to one another, e.g., by hydrogen bonding, ionic bonding, Van der Waals forces, etc. One of skill in the art understands how to couple two molecules such as and IgE Fc and an antigen.

2. Antigen

Disclosed are compositions comprising an Fc portion of IgE coupled to an antigen. The antigen can be an allergen or can be derived from a virus, bacteria, parasite or fungus.

i. Viral Antigen

Disclosed are compositions comprising an Fc portion of IgE coupled to an antigen, wherein the antigen can be derived from a virus. The virus can be selected from the following families: picornaviridae, caliciviridae, togaviridae, flaviviridae, coronaviridae, rhabdoviridae, filoviridae, paramyxoviridae, orthomyxoviridae, bunyaviridae, arenaviridae, reoviridae, retroviridae, hepadnaviridae, parvoviridae, papovaviridae, adenoviridae, herpesviridae, and poxyviridae.

Examples of viruses in these families include but are not limited to Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A (including H1N1 or other Swine H1), Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Examples of specific viral antigens include but are not limited to influenza haemagglutinin (HA), HIV envelop gp120, gp140, gag, HSV glycoprotein D and B, RSV G and F protein. Thus, for example, Fc coupled compositions can comprise an IgE Fc coupled to influenza haemagglutinin (HA), and IgE Fc coupled to HIV envelop gp120, and IgE Fc coupled to gp140, and IgE Fc coupled to gag, an IgE Fc coupled to HSV glycoprotein D, and IgE Fc coupled to glycoprotein B, an IgE Fc coupled to RSV G protein, or an IgE Fc coupled to RSV F protein.

ii. Bacterial Antigen

Disclosed are compositions comprising an Fc portion of IgE coupled to an antigen, wherein the antigen can be derived from a bacterium. The bacterium can be selected from the group consisting of: Escherichia, Klebsiella; Serratia; Pseudomanas; Acinetobacter; Staphylococcus; Enterococcus; Streptococcus; Haemophilus; Neisseria; Bacteroides; Citrobacter; Branhamella; Salmonelia; Shigella; Proteus; Clostridium; Erysipelothrix; Lesteria; Pasteurella; Streptobacillus; Spirillum; Fusospirocheta; Treponema; Borrelia; Actinomycetes; Mycoplasma; Chlamydia; Rickettsia; Spirochaeta; Legionella; Mycobacteria; Ureaplasma; Streptomyces; and Trichomoras.

Examples of specific bacterial species include but are not limited to M. tuberculosis, M. bovis, M. bovis strain BCG, BCG substrains, M. avium, M. intracellulare, M. africanum, M. kansasii, M. marinum, M. ulcerans, M. avium subspecies paratuberculosis, Nocardia asteroides, other Nocardia species, Legionella pneumophila, other Legionella species, Salmonella typhi, other Salmonella species, Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Chlamydia pneumoniae, Chlamydia trachomatis, Chlamydia psittaci, Coxiella burnetti, other Rickettsial species, Ehrlichia species, Staphylococcus aureus, Staphylococcus epidermidis, Streptococcus pneumoniae, Streptococcus pyogenes, Streptococcus agalactiae, Bacillus anthracis, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species.

Examples of specific bacterial antigens include but are not limited to mycobacterium tuberculosis ESAT-6, p85, Streptococcus pneuomonia CbpA, PspA, PsaA, and Chlamydia MOMP. Thus, for example, Fc coupled compositions can comprise IgE Fc coupled to mycobacterium tuberculosis ESAT-6, IgE Fc coupled to p85, IgE Fc coupled to Streptococcus pneuomonia CbpA, IgE Fc coupled to Streptococcus pneuomonia PspA, IgE Fc coupled to Streptococcus pneuomonia PsaA, or IgE Fc coupled to Chlamydia MOMP.

iii. Parasitic Antigen

Disclosed are compositions comprising an Fc portion of IgE coupled to an antigen, wherein the antigen can be derived from a parasite. The parasite can be selected from the group consisting of: Plasmodium, Toxoplasma, Leishmania, Trypanosoma, Schistosoma, Trichinella, Wuchereria, Brugia, Entamoeba, Enterobius, Taenia, Trichomonas, Giardia, Cryptosporidium, Pneumocytis, Babesia, Isospore, Dientamoeba, Onchocerca, Ascaris, Necator, Ancylostoma, Strongyloides, Capillaria, Angiostrongylus, Hymenolepis, Diphyllobothrium, Echinococcus, Paragonimus, Chlonorchis, Opisthorchis, Fasciola, Sarcoptes, Pediculus, Phthirius, and Dermatobia.

Examples of specific parasite species include, but are not limited to, Plasmodium falciparum, P. vivax, P. ovale, P. malaria; Toxoplasma gondii; Leishmania mexicana, L. tropica, L. major, L. aethiopica, L. donovani, Trypanosoma cruzi, T. brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T. saginata, Trichomonas vaginatis, T. hominis, T. tenax; Giardia lamblia; Cryptosporidium parvum; Pneumocytis carinii, Babesia bovis, B. divergens, B. microti, Isospore belli, L. hominis; Dientamoeba fragiles; Onchocerca volvulus; Ascaris lumbricoides; Necator americanis; Ancylostoma duodenale; Strongyloides stercoralis; Capillaria philippinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica Sarcoptes scabiei, Pediculus humanus; Phthirius pubis; and Dermatobia hominis.

Examples of specific parasite antigens include but are not limited to malaria Pfs25, and Trypanosoma cruzi trans-sialidase. Thus, for example, Fc coupled compositions can comprise IgE Fc coupled to malaria Pfs25, or IgE Fc coupled to Trypanosoma cruzi trans-sialidase.

iv. Fungal Antigen

Disclosed are compositions comprising an Fc portion of IgE coupled to an antigen, wherein the antigen can be derived from a fungus. The fungus can be selected from the group consisting of: Cryptococcus; Blastomyces; Aiellomyces; Histoplasfria; Coccidioides; Candidas, Aspergillus, Rhizopus; Rhizomucor; Cunninghammella; Apophysomyces; Sporothrix, Paracoccidioides; Pseudallescheria; Torulopsis; and Dermatophyres.

Examples of fungal species include but are not limited to the following Candidas, Aspergillus and Apophysomyces species: Candidas albicans, C. tropicalis, C. parapsilosis, C. guilliermondii, C. krusei; Aspergillus fumigatus, A. flavus, A. niger; Apophysomyces saksenaea, A. mucor, Cryptococcus neoformans, Histoplama capsulatum, Coccidiodes immitis, Paracoccidiodes brasiliensis, Blastomyces dermitidis, Pneomocystis carnii, Penicillium marneffi, Alternaria alternate, and A. absidia.

Examples of specific fungal antigens include but are not limited to

Candida albicans adhesin rAls3p-N, Sap2, Cryptococcus b-glucan. Thus, for example, Fc coupled compositions can comprise IgE Fc coupled Candida albicans adhesin rAls3p-N, IgE Fc coupled to Sap2, or IgE Fc coupled to Cryptococcus b-glucan.

v. Allergens

Disclosed are compositions comprising an Fc portion of IgE coupled to an antigen, wherein the antigen is an allergen, wherein the allergen can be an antigen derived from pollens, dust, mites, molds, spores, dander, insects or foods. For example, the allergen can be House Dust Mite (Dermatophagoides farina), House Dust Mite (Dermatophagoides pteronyssinus), Food/Storage Mite (Acarus siro), House Dust Mite (Blomia tropicalis), Storage Mite (Chortoglyphus arcuates), House Dust Mite (Euroglyphus maynei), Food/Storage Mite (Lepidoglyphus destructor), Food/Storage Mite (Tyrophagus putrescentiae), House Dust Mite (Glycyphagus domesticus), Bumble Bee Venom (Bombus spp.), European Hornet Venom (Vespa crabro), Honey Bee Venom (Apis mellifera), Mixed Hornet Venom (Dolichovespula spp) Mixed Paper Wasp Venom (Polistes spp.), Mixed Yellow Jacket Venom (Vespula spp.), White (bald)-faced Hornet Venom (Dolichovespula maculate), Yellow Hornet Venom (Dolichovespula arenaria), Carpenter Ant (Camponotus pennsylvanicus), Fire Ant (Solenopsis invicta), Fire Ant (Solenopsis richteri), American Cockroach (Periplaneta Americana), German Cockroach (Blattella germanica), Oriental Cockroach (Blatta orientalis), Horse Fly (Tabanus spp.) House Fly (Musca domestica), Mayfly (Ephemeroptera spp.), Mosquito (Culicidae sp.), Moth (Heterocera spp.), Canary Feathers (Serinus canaria), Cat Epithelia (Felis catus (domesticus)), Cattle Epithelia (Bos Taurus), Chicken Feathers (Gallus gallus (domesticus)), Dog Epithella, (Canis familiaris), Mixed Breeds Duck Feathers (Anas platyrhynchos), Gerbil Epithelia (Meriones unguiculatus), Goat Epithelia (Capra hircus), Goose Feathers (Anser domesticus), Guinea Pig Epithelia (Cavia porcellus (cobaya)), Hamster Epithelia (Mesocricetus auratus), Hog Epithelia (Sus scrofa), Horse Epithelia (Equus caballus), Mouse Epithelia (Mus musculus), Parakeet Feathers (Psittacidae spp.), Pigeon Feathers (Columba fasciata), Rabbit Epithelia (Oryctolagus cuniculus), Rat Spithelia (Rettus norvegicus), Sheep Wool (Ovis aries), Cat Felis catus dander/Antigen (domesticus), Dog Dander (Canis familiaris), Mixed-Breed Poodle Dander (Canis familiaris), Acremonium strictum, Cephalosporium acremonium, Alternaria alternate, Alternaria tenuis, Aspergillus amstelodami, Aspergillus glaucus, Aspergillus flavus, Aspergillus furmigatus, Aspergillus nidulans, Aspergillus niger, Aspergillus terreus, Aspergillus versicolor, Aureobasidium pullulans, Pullularia pullulans, Bipolaris sorokiniana, Drechslera sorokiniana, Helminthosporium sativum, Botrytis cinerea, Candida albicans, Chaetomium globosum, Cladosporium herbarum, Cladosporium sphaerospermum, Hormodendrum hordei, Drechslere spicifera, Curvularia spicifera, Epicoccum nigrum, Epicoccum purpurascens, Epidermophyton floccosum, Fusarium moniliforme, Fusarium solani, Geotrichum candidum, Oospora lactis, Gliocladium viride, Gliocladium deliquescens, Helminthosporium solani, Spondylocladium atrovirens, Microsporum canis, Microsporum lanosum, Mucor mucedo, Mucor circinelloides f. circinelloides, Mucor circinelloides f. lusitanicus, Mucor racemosus, Mucor plumbeus, Mycogone perniciosa, Neurospora intermedia, Neurospora sitophila, Monilia sitophila, Nigrospora oryzae, Paecilomyces variotii, Penicillium brevi-compactum, Penicillium camembertii, Penicillium chrysogenum, Penicillium digitatum, Penicillium expensum, Penicillium notatum, Penicillium roquefortii, Phoma betae, Phomma herbarum, Phoma pigmentivora, Rhigopus oryzae, Rhizopus arrhizus, Rhizopus stolonifer, Rhizopus nigricans Rhodotorula mucilaginosa, Rhodotorula rubra var. mucilaginosa, Saccharomyces cerevisiae, Scopulariopsis brevicaulis, Serpula lacrymans, Merulius lacrymans, Setosphaeria rostrata, Exserohilum rostratum, Helminthosporium halodes, Stemphylium botryosum, Stemphylium solani, Trichoderma harzianum, Trichoderma viride, Trichophyton mentagrophytes, Trichophyton interdigitale, Trichophyton rubrum, Trichothecium roseum, Cephalothecium roseum, Barley Smut (Ustilago nuda ustilago cynodontis), Bermuda Grass Smut Corn Smut (Ustilago maydis) Johnson Grass Smut (Sporisorium cruentum), Oat Smut (Ustilago avenae) Wheat Smut (Ustilago tritici), Bahia (Paspalum notatum) Bermuda (Cynodon dactylon) Blue, Canada (Poa compressa) Brome, Smooth (Bromus inermis), Canary (Phalaris arundinacea), Corn (Zea mays), Couch/Quack (Elytrigia repens (Agropyron repens)), Johnson (Sorghum halepense), Kentucky Blue (Poa pratensis), Meadow Fescue (Festuca pratensis (elatior)) Oat, Cultivated (Avena sativa), Orchard (Dactylis glomerata), Red Top (Agrostis gigantean (alba)), Rye, Cultivated (Secale cereal), Rye, Giant Wild (Leymus (Elymus) condensatus), Rye, Italian (Lolium perenne ssp. Multiflorum), Rye, Perennial (Lolium perenne), Sweet Vernal (Anthoxanehum odoratum), Timothy (Phleum pratense), Velvet (Holcus lanatus) Wheat, Cultivated (Triticum aestivum) Wheatgrass, Western (Elymus (Agropyron) smithii), Allscale (Atriplex polycarpa), Baccharis (Baccharis halimifolia), Baccharis (Baccharis sarothroides), Burrobrush (Hymenoclea salsola), Careless Weed (Amaranthus hybridus), Cocklebur (Xanthium strumarium (commune)), Dock, Yellow (Rumex crispus) Dog Fennel (Eupatorium capillifolium), Goldenrod (Solidago spp.), Hemp, Western Water (Amaranthus tuberculatus (Acnida tamariscina)), Iodine Bush (Allenrolfea occidentalis), Jerusalem Oak (Chenopodium botrys) Kochia/Firebush (Kochia scoparia), Lambs Quarter (Chenopodium album) Marsh Elder, Burweed (Iva xanthifolia) Marsh Elder, Narrowleaf (Iva angustifolia), Marsh Elder, Rough (Iva annua (ciliata)), Mexican Tea (Chenopodium ambrosioides), Mugwort, Common (Artemisia vulgaris), Mugwort, Darkleaved (Artemisia ludoviciana), Nettle (Urtica dioica) Palmer's Amaranth (Amaranthus palmeri), Pigweed, Redroot/Rough (Amaranthus retroflexus), Pigweed, Spiny (Amaranthus spinosus), Plantain, English (Plantago lanceolata), Poverty Weed (Iva axillaris), Quailbrush (Atriplex lentiformis), Rabbit Bush (Ambrosia deltoidea), Ragweed, Desert (Ambrosia dumosa), Ragweed, False (Ambrosia acanthicarpa), Ragweed, Giant (Ambrosia trifida), Ragweed, Short (Ambrosia artemisiifolia), Ragweed, Slender (Ambrosia confertiflora), Ragweed, Southern (Ambrosia bidentata), Ragweed, Western (Ambrosia psilostachya), Russian Thistle (Salsola kali (pestifer)), Sage, Coastal (Artemisia californica), Sage, Pasture (Artemisia frigida), Sagebrush, Common (Artemisia tridentate), Saltbush, Annual (Atriplex wrightii), Shadscale (Atriplex confertifolia), Sorrel, Red/Sheep (Rumex acetosella), Wingscale (Atriplex canescens), Wormwood, Annual, (Artemisia annua), Acacia (Acacia spp.), Alder, European (Alnus glutinosa), Alder, Red (Alnus rubra), Alder, Tag (Alnus incana ssp. Rugosa), Alder, White (Alnus rhombifolia), Ash, Arizona (Fraxinus velutina), Ash, Green/Red (Fraxinus pennsylvanica), Ash, Oregon (Fraxinus latifolia), Ash, White (Fraxinus Americana), Aspen (Populus tremuloides), Bayberry (Myrica cerifera) Beech, American (Fagus grandifolia (americana)), Beefwood/Australian Pine (Casuarina equisetifolia), Birch, Black/Sweet (Betula lenta), Birch, European White (Betula pendula) Birch, Red/River (Betula nigra), Birch, Spring (Betula occidentalis (fontinalis)) Birch, White (Betula populifolia) Box Elder (Acer negundo), Cedar, Japanese (Cryptomeria japonica), Cedar, Mountain (Juniperus ashei (sabinoides)), Cedar, Red (Juniperus virginiana), Cedar, Salt (Tamarix gallica), Cottonwood, Black (Populus balsamifera ssp. Trichocarpa), Cottonwood, Eastern (Populus deltoids), Cottonwood, Fremont (Populus fremontii), Cottonwood, Rio Grande (Populus wislizeni), Cottonwood, Western (Populus monilifera (sargentii)), Cypress, Arizona (Cupressus arizonica), Cypress, Bald (Taxodium distichum), Cypress, Italian (Cupressus sempervirens), Elm, American (Ulmus Americana), Elm, Cedar (Ulmus crassifolia), Elm, Siberian (Ulmus pumila), Eucalyptus (Eucalyptus globulus), Hackberry (Celtis occidentalis), Hazelnut (Corylus Americana), Hazelnut, European (Corylus avellana), Hickory, Pignut (Carya glabra), Hickory, Shagbark (Carya ovate), Hickory, Shellbark (Carya laciniosa), Hickory, White (Carya alba), Juniper, Oneseed (Juniperus monosperma) Juniper, Pinchot (Juniperus pinchotii), Juniper, Rocky Mountain (Juniperus scopulorum), Juniper, Utah (Juniperus osteosperma), Juniper, Western (Juniperus occidentalis), Locust Blossom, Black (Robinia pseudoacacia), Mango Blossom (Mangifera indica), Maple, Coast (Acer macrophyllum), Maple, Red (Acer rubrum), Maple, Silver (Acer saccharinum), Maple, Sugar (Acer saccharum) Melaleuca (Melaleuca quinquenervia (leucadendron)), Mesquite (Prosopis glandulosa (julifiora)), Mulberry, Paper (Broussonetia papyrifera), Mulberry, Red (Moms rubra), Mulberry, White (Morus alba) Oak, Arizona/Gambel (Quercus gambeiji), Oak, Black (Quercus velutina) Oak, Bur (Quercus macrocarpa), Oak, California Black (Quercus kelloggii), Oak, California Live (Quercus agrifolia), Oak, California White/Valley (Quercus lobata), Oak, English (Quercus robur), Oak, Holly (Quercus ilex), Oak, Post (Quercus stellate), Oak, Red (Quercus rubra), Oak, Scrub (Quercus dumosa), Oak, Virginia Live (Quercus virginiana), Oak, Water (Quercus nigra), Oak, Western White/Gany (Quercus garryana), Oak, White (Quercus alba), Olive (Olea europaea), Olive, Russian (Elaeagnus angustifolia) Orange Pollen (Citrus sinensis), Palm, Queen (Arecastrum romanzoffianum (Cocos plumosa)), Pecan (Carya illinoensis), Pepper Tree (Schinus molle), Pepper Tree/Florida Holly (Schinus terebinthifolius), Pine, Loblolly (Pinus taeda) Pine, Eastern White (Pinus strobus), Pine, Longleaf (Pinus palustris), Pine, Ponderosa (Pinus ponderosa), Pine, Slash (Pinus elliottii), Pine, Virginia (Pinus virginiana), Pine, Western White (Pinus monticola), Pine, Yellow (Pinus echinata), Poplar, Lombardy (Populus nigra), Poplar, White (Populus alba), Privet (Ligustrum vulgare), Sweet Gum (Liquidambar styraciflua), Sycamore, Eastern (Platanus occidentalis), Sycamore, Oriental (Platanus orientalis), Sycamore, Western (Platanus racemosa), Sycamore/London Plane (Platanus acerifolia), Walnut, Black (Juglans nigra), Walnut, California Black (Juglans californica), Walnut, English (Juglans regia), Willow, Arroyo (Salix lasiolepis), Willow, Black (Salix nigra), Willow, Pussy (Salix discolor), Daisy, Ox-Eye (Chrysanthemum leucanthemum), Dandelion (Taraxacum officinale), Sunflower (Helianthus annuus), Alfalfa (Medicago sativa), Castor Bean (Ricinus communis), Clover, Red (Trifolium pratense), Mustard (Brassica spp.), Sugar Beet (Beta vulgaris), Almond (Prunus dulcis), Apple (Malus pumila), Apricot (Prunus armeniaca), Banana (Musa paradisiaca (sapientum)), Barley (Hordeum vulgare), Bean, Lima (Phaseolus lunatus), Bean, Navy (Phaseolus vulgaris), Bean, Pinto (Phaseolus sp.), Bean, Red Kidney (Phaseolus sp.), Bean, String/Green (Phaseolus vulgaris), Blackberry (Rubus allegheniensis), Blueberry (Vaccinium sp.), Broccoli (Brassica oleracea var. botrytis), Buckwheat (Fagopyrum esculentum), Cabbage (Brassica oleracea var. capitata), Cacao Bean (Theobroma cacao), Cantaloupe (Cucumis melo). Carrot (Daucus carota). Cauliflower (Brassica oleracea var. botrytis), Celery (Apium graveolens var. dulce), Chemy (Prunus sp.), Cinnamon (Cinnamomum verum), Coffee (Coffee Arabica), Corn (Zea mays), Cranberry (Vaccinium macrocarpon), Cucumber (Cucumis sativus), Garlic (Allium sativum), Ginger (Zingiber officinale), Grape (Vitis sp.), Grapefruit (Citrus paradise), Hops (Humulus lupulus). Lemon (Citrus limon), Lettuce (Lactuca sativa), Malt Mushroom (Agaricus campestris), Mustard (Brassica sp.), Nutmeg (Myristica fragrans), Oat (Avena sativa), Olive, Green (Olea europaea), Onion (Allium cepa var. cepa). Orange (Citrus sinensis). Pea, Blackeye (Vigna unguiculata), Pea, Green (English) (Pisum sativum), Peach (Prunus persica), Pear (Pyrus communis), Pepper, Black (Piper nigrum) Pepper, Green (Capsicum annuum var. annuum), Pineapple (Ananas comosus), Potato, Sweet (Ipomoea batatas), Potato, White (Solanum tuberosum), Raspberry (Rubus idaeus var. idaeus), Rice (Oryza sativa). Rye (Secale cereal), Sesame Seed (Sesamum orientale (indicum)), Soybean (Glycine max), Spinach (Spinacia oleracea), Squash, Yellow (Cucurbita pepo var. melopepo), Strawberry (Fragaria chiloensis), Tomato (Lycopersicon esculentum (lycopersicum)), Turnip (Brassica rapa var. rapa), Vanilla Bean (Vanilla planifolia), Watermelon (Citrullus lanatus var. lanatus), Wheat, Whole (Triticum aestivum), Bass, Black (Micropterus sp.), Catfish (Ictalurus punctatus), Clam (Mercenaria mercenaria), Codfish (Gadus morhua), Crab (Callinectes sapidus), Flounder (Platichthys sp.), Halibut (Hippoglossus sp.), Lobster (Homarus americanus), Mackerel (Scomber scombrus), Oyster (Crassostrea virginica), Perch (Sebastes marinus), Salmon (Salmo salar), Sardine (Clupeiformes), Scallop (Pectan magellanicus), Shrimp (Penaeus sp.), Trout, Lake (Salvelinus sp.), Tuna Fish (Thunnus sp.), Beef (Bos Taurus), Lamb (Ovis aries), Pork (Sus scrofa), Chicken (Gallus gallus), Egg, Chicken, (Gallus gallus), White Egg, Chicken, (Gallus gallus), Yolk Turkey (Meleagris gallopavo) Casein, bovine (Bos Taurus), Milk, bovine (Bos Taurus), Brazil Nut (Bertholletia excels), Cashew Nut (Anacardium occidentale), Coconut (Cocos nucifera), Filbert/Hazelnut (Corylus Americana), Peanut (Arachis hypogaea), Pecan (Carya illinoensis), Walnut, Black (Juglans nigra), Walnut, English (Juglans regia), and Latex.

In some aspects, the allergen can be urushiols (pentadecylcatechol or heptadecylcatechol) or sesquiterpenoid lactones. The urushiols can be from Toxicodendron species that include but are not limited to poison ivy, poison oak and poison sumac. The sesquiterpenoid lactones can be from ragweed or related plants.

Thus, for example, Fc coupled compositions can comprise IgE Fc coupled dust mite, IgE Fc coupled to bee venom, IgE Fc coupled to peanut allergen, IgE Fc coupled to shellfish allergen, IgE Fc coupled to a pollen allergen, or IgE Fc coupled to a mold allergen.

vi. Tumor Antigen

Disclosed are compositions comprising an Fc portion of IgE coupled to an antigen, wherein the antigen can be a tumor antigen. Tumor antigens can be but are not limited to alpha (a)—fetoprotein; adenocarcinoma antigen recognized by T cells 4; B antigen; b-catenin/mutated; breakpoint cluster region-Abelson (bcr-abl); CTL-recognized antigen on melanoma; carcinoembryonic antigen peptide—1; p53, caspase-8; cell-division cycle 27 mutated; cycline-dependent kinase 4 mutated; carcinoembryonic antigen; cancer/testis (antigen); cyclophilin B; differentiation antigen melanoma; elongation factor 2 mutated; Ets variant gene 6/acute myeloid leukemia 1 gene ETS; glycoprotein 250; G antigen; N-acetylglucosaminyltransferase V; glycoprotein 100 kD; helicose antigen; human epidermal receptor-2/neurological; human papilloma virus E7; heat shock protein 70-2 mutated; human signet ring tumor—2; human telomerase reverse transcriptase; intestinal carboxyl esterase; L antigen; low density lipid receptor/GDP-L-fucose: b-D-galactosidase 2-a-Lfucosyltransferase; melanoma antigen; melanoma antigen recognized by T cells-1/Melanoma antigen A; melanocortin 1 receptor; myosin mutated; mucin 1; melanoma ubiquitous mutated 1, 2, 3; protein 15; protein of 190 KD bcr-abl; promyelocytic leukaemia/retinoic acid receptor a; preferentially expressed antigen of melanoma; prostate-specific antigen; prostate-specific membrane antigen; renal antigen; renal ubiquitous 1 or 2; sarcoma antigen; squamous antigen rejecting tumor 1 or 3; translocation Ets-family leukemia/acute myeloid leukemia 1; triosephosphate isomerase mutated; tyrosinase related protein 1, or gp75; tyrosinase related protein 2; TRP-2/intron 2; Wilms' tumor gene; ras; surviving; her2; CD20, mucin-1, and MAGE-3.

Thus, for example, Fc coupled compositions can comprise IgE Fc coupled to her2, IgE Fc coupled to p53, IgE Fc coupled to mucin-1, IgE Fc coupled to caspase-8. These examples allow one of skill in the art to understand that IgE Fc coupled to any of the known tumor antigens is contemplated.

vii. Cellular Antigen

Disclosed are compositions comprising an Fc portion of IgE coupled to an antigen, wherein the antigen can be a cell surface, cytoplasmic, nuclear, or mitochondrial antigen.

Examples of cell surface, cytoplasmic, nuclear, or mitochondrial antigens include, but are not limited to, DNA, RNA, human epidermal receptor-2, cyclophilin B, human telomerase reverse transcriptase, p32/C1QBP. Thus, for example, Fc coupled compositions can comprise IgE Fc coupled to human epidermal receptor-2, IgE Fc coupled to cyclophilin B, IgE Fc coupled to human telomerase reverse transcriptase, or IgE Fc coupled to p32/C1QBP.

3. Immunotherapeutic Agent

Disclosed are compositions comprising an Fc portion of IgE coupled to an immunotherapeutic agent. The immunotherapeutic agent can be any immunotherapeutic agent known to treat airway inflammation. Immunotherapeutic agents can be anti-inflammatory agents or cell-signaling agents. The following listed immunotherapeutic agent call all be used to treat airway inflammation.

Disclosed are compositions comprising an Fc portion of IgE coupled to an immunotherapeutic agent, wherein the immunotherapeutic agent can be but is not limited to Cytotoxic T lymphocyte antigen 4 (CTLA4), Soluble IL-4 receptor, Soluble IL-13 receptor, IL-5R, Thymic stromal derived lymphopoietin receptor (TSLPR), IL10, IL-9 receptors, IL-17 receptors, IL-21 receptors, IL-25 receptors, IL-31 receptors, IL-33 receptors, transforming growth factor beta (TGFbeta), TGFbeta receptors, Histamine receptors, Prostaglandin receptors, FcepsilonRI alpha, programmed death 1 (PD1), Flt3-ligand, leukotriene receptor, Tumor necrosis factor (TNFR), LIGHT receptor, OX40L, IL-1beta receptor, c-kit, ADAM, soluble intercellular adhesion molecule 1 (sICAM-1), soluble IL-2R, CD48, Pulmonary surfactant protein D (SPD), soluble β2-Adrenergic receptor, B7-1, B7-2, B7-H1, B7-H2, leukocyte-associated immunoglobulin-like receptor 1 (LAIR1), lymphocyte activation gene 3 protein (LAG3), CD160, LAG3, lymphocyte activation gene 3 protein (TIGIT), type I transmembrane (or T cell) immunoglobulin and mucin. (TIM3), or B and T lymphocyte attenuator (BTLA).

4. Vaccines

Disclosed are vaccines comprising a composition comprising an Fc portion of IgE coupled to an antigen. The term “vaccine,” as used herein, is defined as a composition used to provoke an immune response and confer immunity, at least briefly, after administration of the composition to a subject. It is understood and herein contemplated that vaccines can be therapeutic or prophylactic. The vaccines elicit an immune response to the antigen and provide both prophylactic and therapeutic purposes.

In some aspects, the vaccines can comprise more than one Fc portion of IgE coupled to an antigen. The more than one Fc portion of IgE coupled to an antigen can be formulated in the same composition or separate compositions.

5. Pharmaceutical Compositions

Disclosed are compositions comprising an Fc portion of IgE coupled to an antigen further comprising a pharmaceutically acceptable carrier. Also disclosed are compositions comprising an Fc portion of IgE coupled to an immunotherapeutic agent further comprising a pharmaceutically acceptable carrier. Compositions comprising a pharmaceutically acceptable carrier are pharmaceutical compositions.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration.

i. Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

ii. Therapeutic Uses

1. Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. A typical daily dosage of an IgE conjugated to an antigen used alone might range from about 1 μg/kg to up to 100 mg/kg of body weight or more per day, depending on the factors mentioned above.

6. Combinations

Disclosed are compositions and pharmaceutical compositions comprising more than one of the disclosed IgE Fc coupled compositions. For example, a composition can comprise IgE Fc coupled to a viral antigen and IgE Fc coupled to a fungal antigen. In some aspects the composition can comprise IgE Fc coupled to CTLA4 and IgE Fc coupled to TNFR. In some aspects a composition can comprise an IgE Fc coupled to an antigen and an IgE Fc coupled to an immunotherapeutic.

### B. METHODS OF TREATING OR PREVENTING AIRWAY INFLAMMATION

Disclosed are methods of treating airway inflammation comprising administering to a subject an effective amount of any of the disclosed IgE Fc coupled compositions. For example, the Fc portion of IgE coupled to an antigen or the Fc portion of IgE coupled to an immunotherapeutic can be used to treat airway inflammation. Treating refers to partially or completely alleviating, ameliorating, relieving, delaying onset of, inhibiting progression of, reducing severity of, and/or reducing incidence of one or more symptoms or features of a particular disease, disorder, and/or condition. The treatment can be any reduction in severity, progression, or incidence of a disease or condition including but not limited to complete ablation of the disease, condition, or the symptoms of the disease or condition. Therefore, in the disclosed methods, “treatment” can refer to a 10%, 20%, 30%, 40%, 50%, 60%, 70%, 80%, 90%, or 100% reduction in the severity, progression, or incidence of a disease or condition. For example, “treating” airway inflammation can refer to inhibiting or reducing airway inflammation. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of a vaccine to a subject.

Methods of treating airway inflammation by administering an effective amount of the Fc portion of IgE coupled to an antigen can work in a prophylactic or preventative manner. The IgE Fc can transport the antigen of interest, specifically an antigen prominent in airway inflammation, across the epithelial cells allowing for an immune response to be triggered in response to the antigen. This triggered immune response can then prevent airway inflammation by fighting off the infection or allergen responsible for causing the inflammation. For example, an individual with an allergy to pollen can be administered a composition comprising the Fc portion of IgE coupled to a particular pollen antigen. Transport of the Fc portion of IgE coupled to the particular pollen antigen across the respiratory epithelial cells can trigger an immune cell activation of cells to target that pollen antigen. Therefore, upon inhalation of pollen, the activated immune cells will work to prevent airway inflammation.

Airway inflammation can be from asthma, allergies, or infection. Thus, the methods of treating airway inflammation can be tailored to each individual based on their primary source of inflammation.

Methods of treating airway inflammation can include administering the disclosed IgE Fc coupled compositions to those subjects at risk for developing airway inflammation. For example, subjects at risk for developing asthma include those individuals with a family history of allergic disease, the presence of allergen-specific IgE, viral respiratory illnesses, exposure to aeroallergens, exposure to cigarette smoke, and obesity.

Methods of treating airway inflammation with the disclosed compositions can comprise intranasal or oral administration.

### C. METHODS OF INTERFERING WITH TH2 CELL ACTIVATION

Disclosed are methods of interfering with T cell activation comprising administering to a subject an effective amount a compositions comprising an Fc portion of IgE coupled to an immunotherapeutic. The T cell activation can occur in the airway.

The immunotherapeutic can be but is not limited to Cytotoxic T lymphocyte antigen 4 (CTLA4), Soluble IL-4 receptor, Soluble IL-13 receptor, IL-5R, Thymic stromal derived lymphopoietin receptor (TSLPR), IL10, IL-9 receptors, IL-17 receptors, IL-25 receptors, IL-31 receptors, IL-33 receptors, transforming growth factor beta (TGFbeta), Histamine receptors, Prostaglandin receptors, FcepsilonRI alpha, programmed death 1 (PD1), Flt3-ligand, leukotriene receptor, Tumor necrosis factor (TNFR), LIGHT receptor, OX40L, IL-1beta receptor, c-kit, ADAM, soluble intercellular adhesion molecule 1 (sICAM-1), soluble IL-2R, CD48, Pulmonary surfactant protein D (SPD), soluble β2-Adrenergic receptor, B7-1, B7-H1, leukocyte-associated immunoglobulin-like receptor 1 (LAIR1), lymphocyte activation gene 3 protein (LAG3), CD160, LAG3, lymphocyte activation gene 3 protein (TIGIT), type I transmembrane (or T cell) immunoglobulin and mucin. (TIM3), or B and T lymphocyte attenuator (BTLA).

In some aspects, methods of interfering with Th2 cell activation comprising administering to a subject an effective amount a compositions comprising an Fc portion of IgE coupled to an immunotherapeutic, wherein the immunotherapeutic can be CTLA4. The use of CTLA4 allows for the composition to block signaling through a CD28 molecule on the T cell.

Methods of interfering with Th2 cell activation by administering an effective amount composition comprising an Fc portion of IgE coupled to an immunotherapeutic can directly or indirectly interfere with Th2 activation. An immunotherapeutic that blocks or interferes with the signals from an antigen presenting cell can be conjugated to the IgE Fc and interfere with Th2 activation.

Methods of interfering with T cell activation with the disclosed compositions can comprise intranasal or oral administration.

### D. METHODS OF BLOCKING MAST CELL DEGRANULATION

Disclosed are methods of blocking mast cell degranulation comprising administering to a subject an effective amount of a composition comprising an Fc portion of IgE coupled to an antigen or coupled to an immunotherapeutic.

IgE receptors are present on mast cells. IgE bound to the IgE receptors on mast cells can be cross-linked by antigens. This triggers the degranulation of mast cells which releases potent inflammatory mediators, such as but not limited to histamine, lipid mediators, and proteases. Therefore, the disclosed compositions comprising an Fc portion of IgE coupled to an antigen or immunotherapeutic can bind to Fc receptors on the surface of mast cells, prevent IgE from binding to the Fc receptors, and prevent or reduce degranulation.

Methods of blocking mast cell degranulation with the disclosed compositions can comprise intranasal or oral administration.

### E. METHODS OF MODULATING AN IMMUNE RESPONSE

Disclosed are methods of modulating the immune system comprising administering to a subject an effect amount of a composition comprising the Fc portion of IgE coupled to an antigen or an immunotherapeutic. In some aspects, modulating the immune system can comprise inducing an immune response. In some aspects, modulating the immune system can comprise reducing an immune response.

Inducing the immune response can occur when the composition acts as a vaccine. Administering a composition comprising the Fc portion of IgE coupled to an antigen can lead to triggering of an immune response to the antigen. Thus, the composition can elicit an immune response to the antigen.

Reducing or blocking the immune response can occur when the composition acts as an immunotherapy. Administering a composition comprising the Fc portion of IgE coupled to an immunotherapeutic can result in blocking or interfering with cell signaling and thus preventing or limiting an immune response.

Disclosed are methods of modulating the immune system comprising administering to a subject an effect amount of a composition comprising the Fc portion of IgE coupled to an antigen or an immunotherapeutic, wherein the composition further comprises an adjuvant. For example, a composition comprising the Fc portion of IgE coupled to an antigen can further comprise an adjuvant to help prime or booster the immune response.

Disclosed are methods of decreasing proinflammatory cytokines in the bronchoalveolar lavage fluid (BALF) comprising administering to a subject an effective amount a composition comprising the Fc portion of IgE coupled to an immunotherapeutic. These methods can result in a reduction or decrease in the immune response.

Disclosed are methods of reducing the numbers of hematopoietic cells in the BALF comprising administering to a subject an effective amount of a composition of comprising the Fc portion of IgE coupled to an immunotherapeutic. Hematopoietic cells can be eosinophils, macrophages, neutrophils, or lymphocytes.

Methods of modulating the immune system with the disclosed compositions can comprise intranasal or oral administration.

### F. METHODS OF DELIVERING A POLYPEPTIDE ACROSS AIRWAY EPITHELIAL CELLS

Disclosed are methods of delivering an antigen across airway epithelial cells comprising administering to a subject an effect amount of the disclosed compositions comprising the Fc portion of IgE coupled to an antigen.

Disclosed are methods of delivering an immunotherapeutic across airway epithelial cells comprising administering to a subject an effect amount of the disclosed compositions comprising the Fc portion of IgE coupled to an antigen.

The Fc portion of IgE can bind to CD23 on airway epithelial cells and is then transported across the epithelial cells. Thus, if the Fc portion of IgE is coupled to another agent, it can be used to transport that agent across the airway epithelial cells.

### G. COMBINATIONS

The disclosed methods include administering an effective amount of an IgE Fc coupled to an antigen or immunotherapeutic. These methods can also involve

combination therapies wherein more than one IgE Fc coupled to an antigen or immunotherapeutic is administered to a subject. For example, the methods of treating airway inflammation can involve administering a composition having one or more IgE Fc coupled compositions. The one or more IgE Fc coupled compositions can be coupled to different antigens. For example, the methods can include administering a composition having an IgE Fc coupled to CTLA4 and an IgE Fc coupled to an interleukin. The one or more IgE Fc coupled compositions can be formulated separately or together.

The disclosed methods can also be used in combination. For example, the methods of treating airway inflammation can be performed in combination with the methods of modulating an immune response.

### H. DELIVERY

The delivery or administration can include intranasal or oral administration. Examples include but are not limited to topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

### I. KITS

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for preparing compositions comprising IgE Fc coupled to an antigen or an immunotherapeutic, the kit comprising IgE Fc. The kits also can contain the antigens or immunotherapeutics of interest.

### J. DEFINITIONS

The term “modulate” refers to an increase or decrease over normal levels. For example, modulating the immune system with the disclosed compositions means increasing or decreasing the immune response compared to the immune response prior to administration of the composition.

As used herein, the term “subject” refers to any organism to which the disclosed compositions can be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; zoo and wild animals). Typically, “subjects” are animals, including mammals such as humans and primates; and the like. Subjects can also refer to a cell or a cell line.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Those skilled in the art will recognize, or be able to ascertain using no more than routine experimentation, many equivalents to the specific embodiments of the method and compositions described herein. Such equivalents are intended to be encompassed by the following claims.

### EXAMPLES

### K. EXAMPLE 1

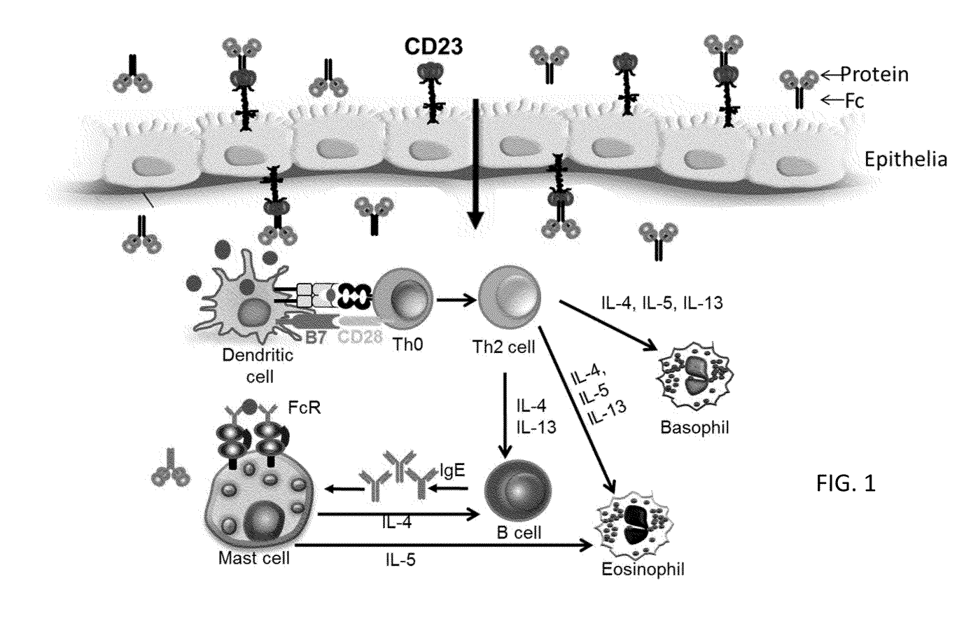

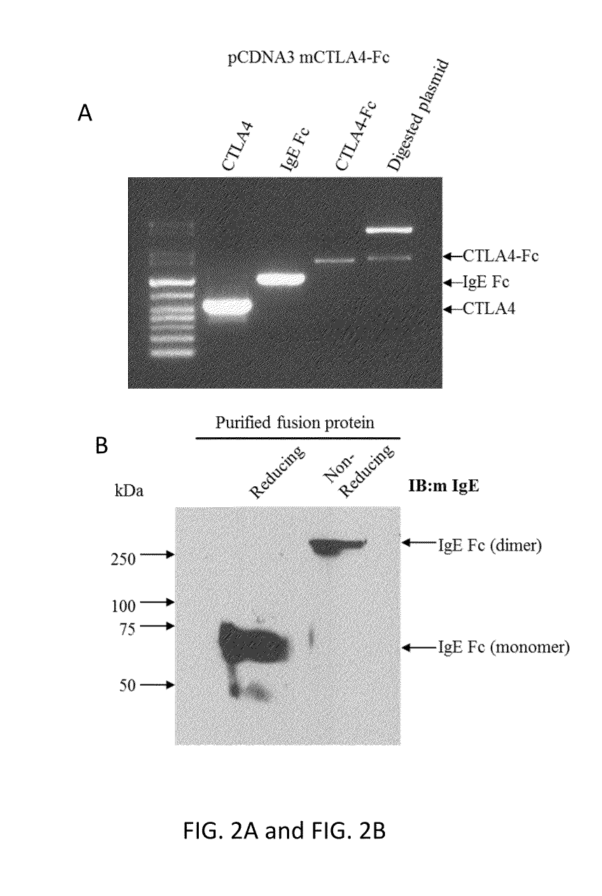

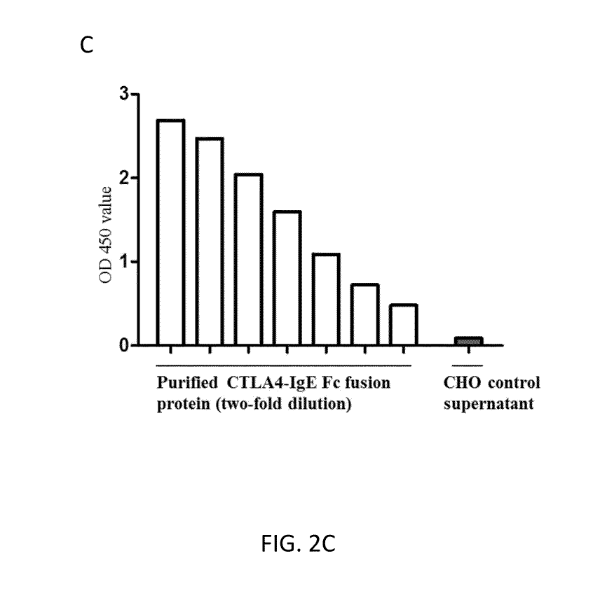

IgE Fc coupled compositions can be transported to the airway via CD23 mediated delivery. FIG. 1 is a schematic diagram of the transport mechanism and the cells present in the airway that can be affected by the IgE Fc coupled compositions.

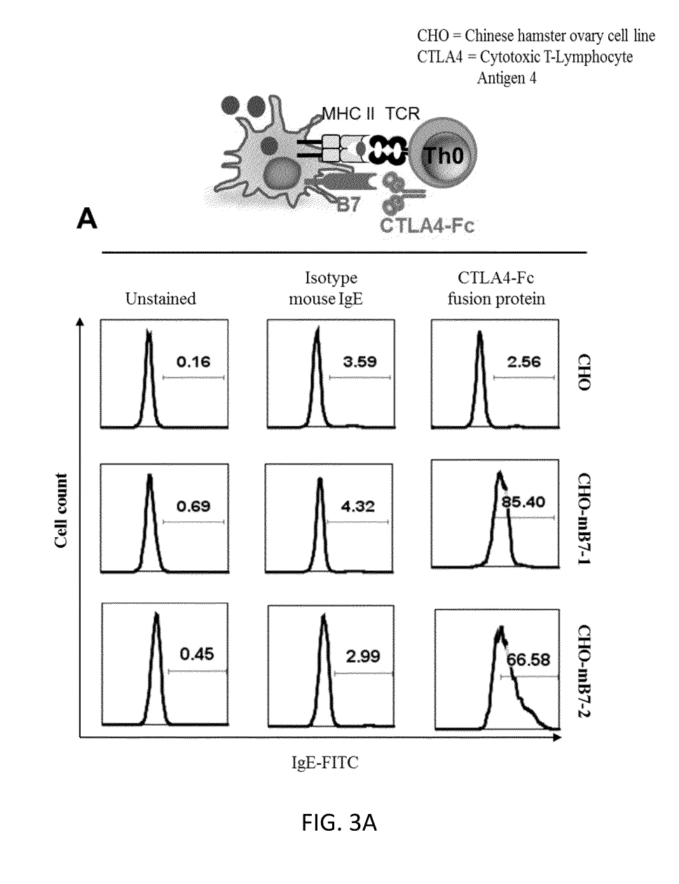

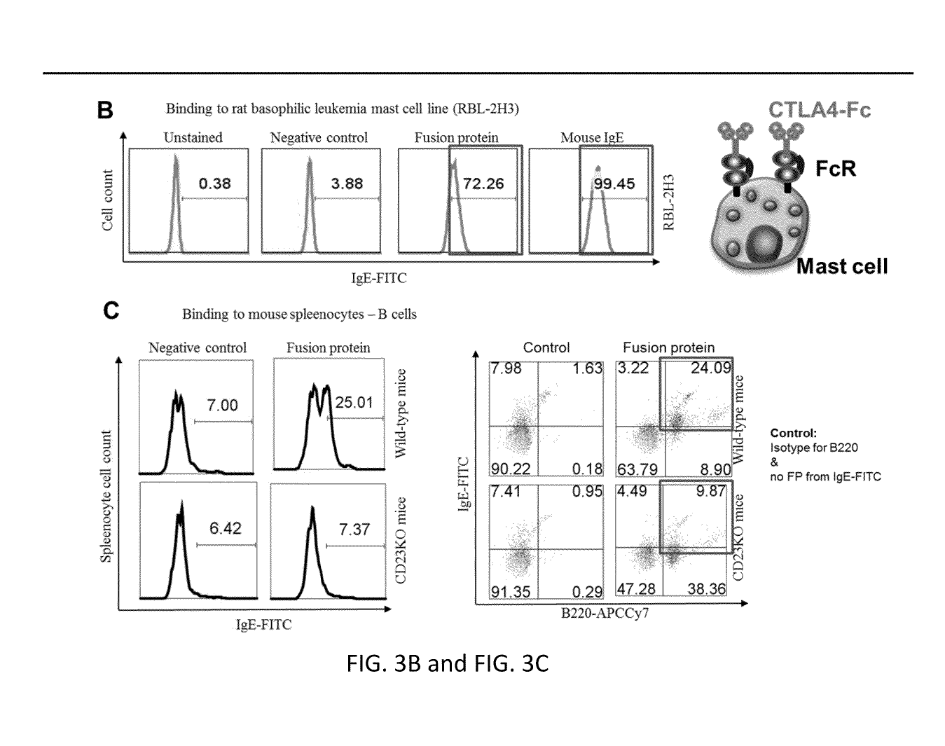

FIG. 3 shows that recombinant CTLA4-Fc significantly interacts with either B7-1 or B7-2 molecules in CHO transfected cell lines in comparison with CHO cell line or control staining with normal IgG. Thus, CTLA4, when coupled to IgE Fc, can be used to block the binding of B7-1 and B7-2 to their normal ligands.

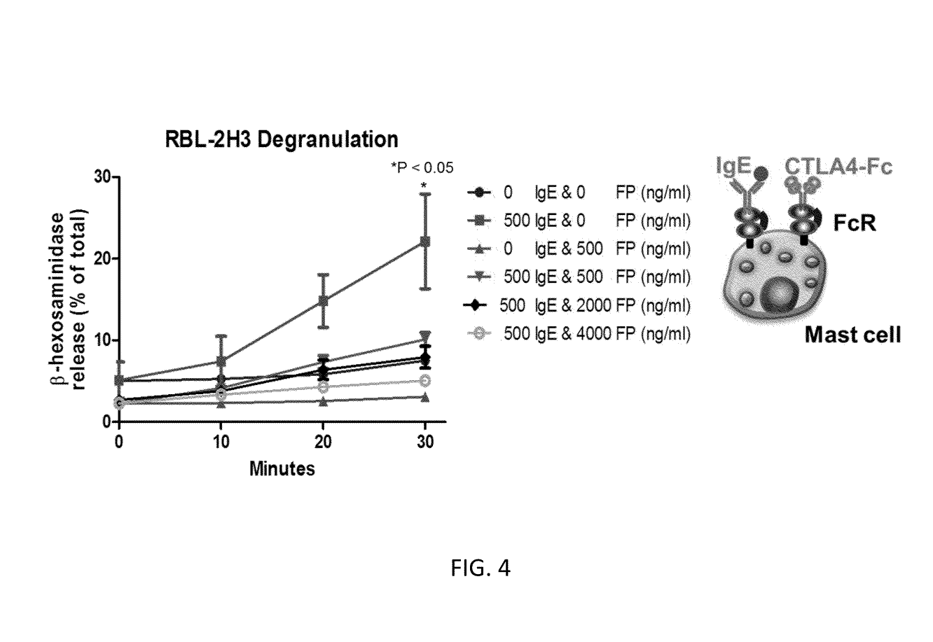

Not only can CTLA-4-Fc block the interaction of B7 to its normal ligand, but CTLA4-Fc can bind to Fc receptors on mast cells and prevent or reduce degranulation (FIG. 4).

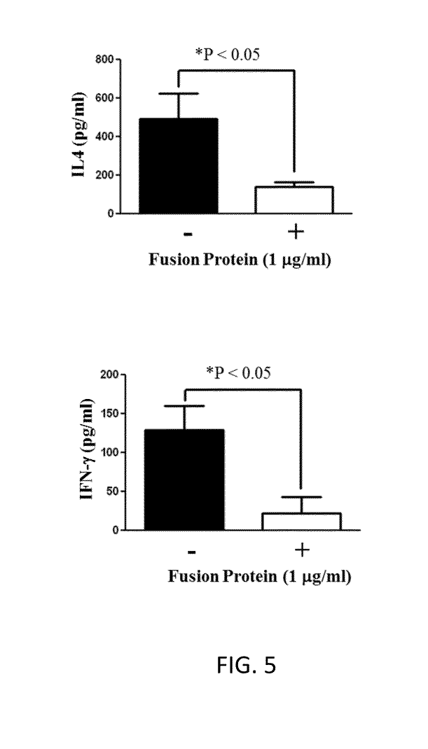

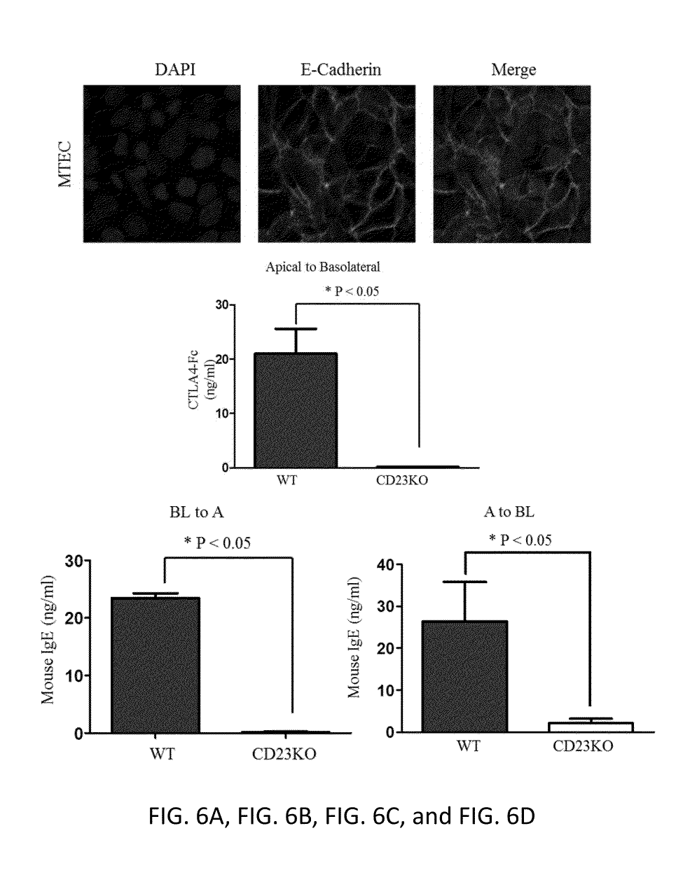

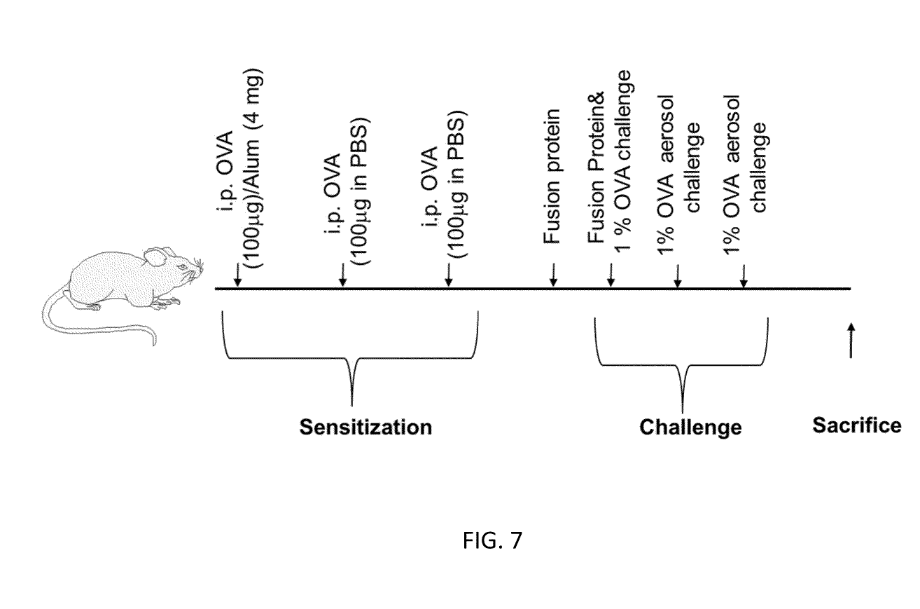

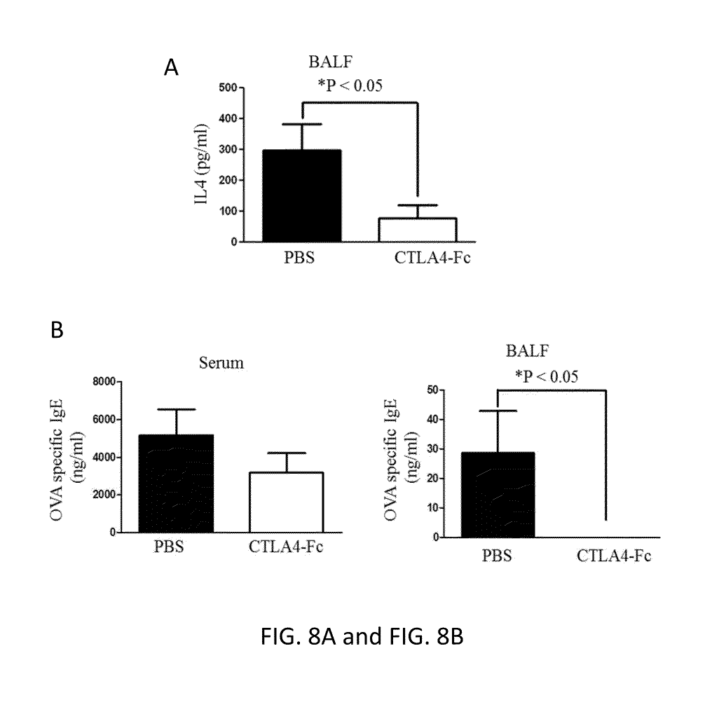

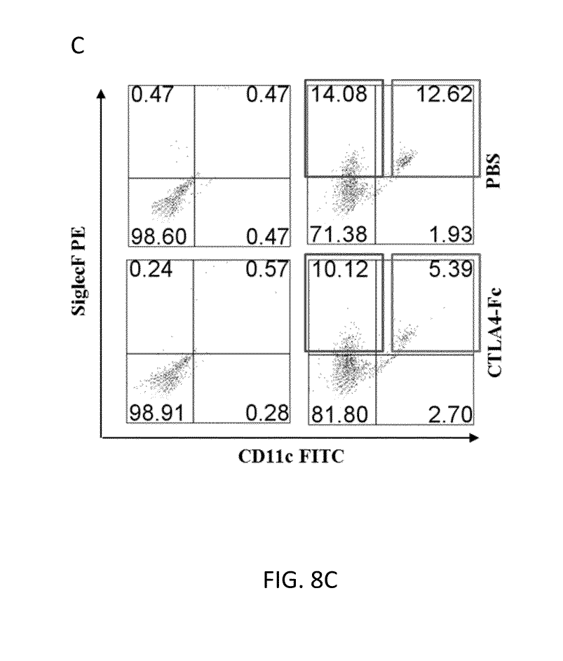

Proinflammatory cytokines, such as IL-4 and IFN-γ, can be decreased in the presence of CTLA4-Fc (FIG. 5). The presence of CTLA4-Fc can have a therapeutic effect in a mouse asthma model (FIG. 8).

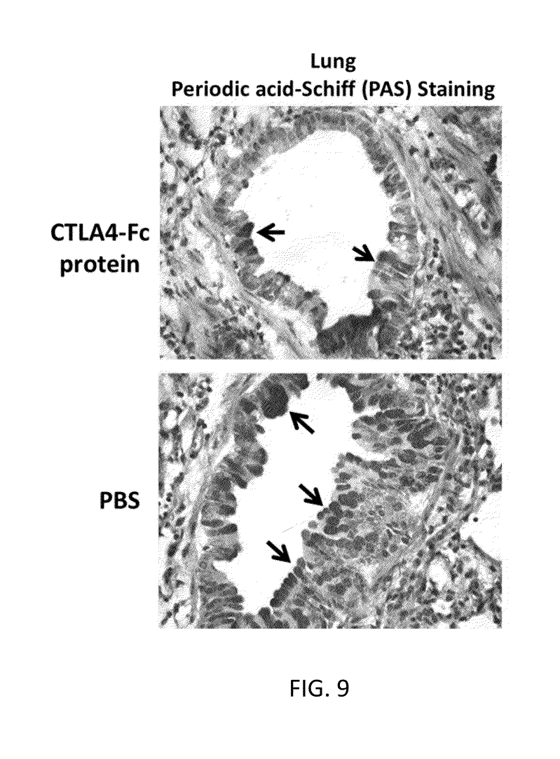

Infiltration of leukocytes in histologic sections of lungs from OVA-challenged control mice were found and lung tissue sections from OVA-challenged mice showed a distinct inflammatory infiltrate and erosion in peribronchial areas (FIG. 9). In contrast, histological sections from CTLA4-Fc fusion protein-treated mice indicated reduced airway inflammation in lung tissue. The OVA-challenged/PBS-treated control mice significantly increased the numbers of PAS-positive cells (Arrow) when compared with OVA-challenged/CTLA4-Fc treated mice.

### L. EXAMPLE 2

Epithelial monolayers lining the respiratory tracts are impervious to macromolecule diffusion in the absence of inflammation, due to the presence of intercellular tight junctions at the apical poles. The tight junctions divide polarized airway epithelial cells into apical and basolateral domains. These domains further form mucosal barriers that allow for the selective exchange of macromolecules between the lumen and submucosal tissue under physiological conditions. Therefore, soluble macromolecules, such as immunotherapeutic proteins, across the mucosal epithelium are generally blocked by mucosal barrier. However, crossing of the macromolecules over the mucosal barrier can be mediated by a transcellular transport pathway, or transcytosis. Specifically, the transcellular pathway involves endocytic uptake of macromolecules, specifically by receptor-mediated and/or fluid-phase endocytosis, at the apical or basolateral membrane. The molecules are then transported through the cell in endocytic vesicles to the opposite membrane surface, where they are released into the lumen or submucosal space. Beneath the epithelial lining, lymphocytes, mast cells, basophils, eosinophils, and dendritic cells accumulate in either a loosely-infiltrated fashion or an organized fashion forming the organized bronchus associated lymphoid tissue (BALT). Therefore, the transcellular pathway is a major route in moving soluble macromolecules across the airway epithelial barrier and initiates a cross-talk between immunotherapeutic agents and the immune cells.

IgE is present in airway secretions; its level can be enhanced in persons who have allergic rhinitis or bronchial asthma. For many years, the presence of IgE in airway secretions has been simply believed to be transduced passively from the serum. The complete paucity of mechanistic studies of IgE transport in the airway has seriously hampered our understanding of allergic inflammation in the lung. Our recent study has found that human and murine respiratory epithelial cells express CD23, a receptor for the Fc portion of IgE. In comparison with high affinity IgE receptor FccR1, CD23 was considered as a low affinity receptor for a long time. However, recent studies show that CD23 forms a trimer that allows its lectin domains to come within close proximity of each other and to cooperate in binding one IgE molecule, consequently resulting in CD23's high affinity binding to IgE antibody. Studies have shown that CD23 was expressed in human airway epithelial cells. Most importantly, CD23 was functionally capable of transporting IgE antibody across human lung and bronchial epithelial cell lines or primary bronchial epithelial tissues. Furthermore, this study has demonstrated that mouse IgE was transported across airway mucosal barrier in wild-type, but not CD23 knockout, mice when biotin-labeled IgE molecules were intranasally administrated. Since airway epithelial cells are the first cell layer to come into contact with inhaled immunotherapeutic proteins, CD23 can be used to deliver an immunotherapeutic protein. If fused to an IgE Fc fragment, across the airway barrier to gain access to underlying immune effector cells, consequently modulating or dampening the inflammations and hypersensitivity responses in the airway. Therefore, CD23 mediated transport of IgE Fc-fused immunotherapeutic proteins in the airway tract, can be integrated into therapeutic interventions for airway allergic inflammation.

1. CD23 to Deliver IgE Fc-Fused Immunotherapeutic Proteins Across the Airway Barrier.

Helper Th2 cells and/or their secreted effector molecules mediate the immune response to allergens and are triggered by exposure to specific allergens leading to allergic asthma. Thus, inhibiting or eliminating Th2 cells is a beneficial strategy for treating asthma as long as generalized immunosuppression is avoided. Cytotoxic T-lymphocyte antigen 4 (CTLA-4) is a negative regulator of T-cell activation, and its inhibitory effects can be accomplished by competition with CD28 for binding to B7-1 or B7-2 with a much high affinity on antigen-presenting cells (APCs). Recombinant mouse CTLA4-Fc (IgE) proteins have been produced. The affinity purified fusion proteins can be intranasally administered into the airway tract of wild-type and CD23-KO mice and thus analyze its specific transport. The impact of fusion protein transport on airway inflammation and hyper-reactivity can be assessed in a ovalbumin (OVA)-based murine asthma model. A chimeric mouse created between wild-type and CD23 KO mice can be used to show the specific transport function of epithelial CD23 in blocking inflammation initiation and development. The subsequent allergic inflammation in the lung can be evaluated by immunological parameters and histopathology.

Airway inflammation and dysfunction is one of the most important human diseases. Through a previously unrecognized CD23-mediated IgE transport in the airway epithelial cells, CD23-mediated transport of immunotherapeutic proteins can be an intervening strategy for the allergic inflammation. This study offers effective treatment options for patients suffering with asthma and other allergic diseases, such as asthma and chronic obstructive pulmonary disease.

Asthma is a serious chronic inflammatory lung disease characterized by recurrent episodes of wheezy labored breathing accompanied by dry coughing and viscous mucus. These symptoms result from bronchoconstriction, bronchial mucosal thickening by edema, eosinophilic infiltration, bronchial wall remodeling and excessive mucus production with plugging of the conducting airways in the lungs. These airway changes lead to increased bronchial hyperreactivity. Asthma affects approximately 300 million people worldwide and can be fatal. Atopic or allergic asthma generally occurs in childhood or young adulthood (under the age of 40) and is caused by common allergens e.g. pollens, house dust. The most severe chronic refractory asthma accounts for 5-10% of adults with asthma and is characterized by persistent symptoms and frequent exacerbations, despite treatment with high dose inhaled and/or oral corticosteroids. These patients are at greater risk of fatal and near-fatal exacerbations and display serious symptoms, resulting in a considerable impact on quality of life.

Asthma represents an extremely formidable challenge for traditional therapy due to its complicated biology. It starts with the activation of allergen-specific Th2 cells by antigen presenting cells (APCs) followed by their proliferation, cytokine production and the emergence of memory cells. The resulting immunopathological response causes airway obstruction and eventual lung damage. The disease is currently treated and, more or less, controlled depending on severity, with anti-inflammatory drugs such as corticosteroids (inhaled or oral), leukotriene modifiers, theophylline, anti-IgE therapy, etc. Unfortunately, none of these treatments are curative and some asthmatic patients do not respond to intense anti-inflammatory therapies. Additionally, the use of long-term steroids leads to many undesired side effects. For this reason, novel and more effective immunotherapeutic strategies are greatly needed. The reduction or elimination of allergen-specific Th2 cells in early phase of disease can reduce the consequences of repeated allergic inflammatory responses such as lung remodeling without causing generalized immunosuppression.

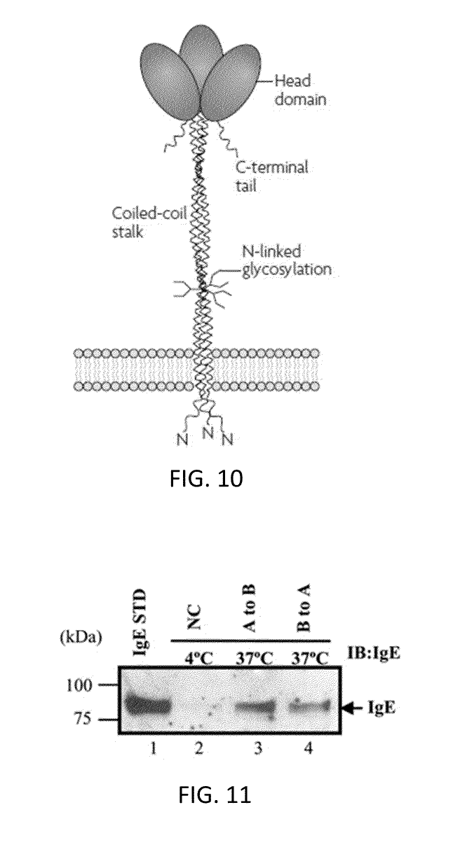

In order for proteins with immunotherapeutic function to interfere with the functions of CD4 Th2 cells, they must cross the epithelial monolayer lining the airway tract. However, these polarized epithelial cells are impervious to macromolecule diffusion due to the presence of tight junctions at the apical poles. Fortunately, a transcellular transport pathway, also called transcytosis, exists to allow efficient transfer of macromolecule proteins across polarized airway epithelium. In this nature transfer pathway, the molecules are protected and transported through the cell in endocytic vesicles to the opposite membrane surface, where they are released into the extracellular space. It was shown that IgE can be transported from the apical to the basolateral surface within polarized airway epithelia by CD23 molecule. Herein we investigate using CD23 to transport immunotherapeutic proteins, if fused with the Fc portion of IgE, across the airway mucosal barrier to gain access to underlying immune effector cells. Notably, although CD23 is previously called low affinity receptor for IgE relative to high affinity receptor FcεRI, several studies found that CD23's trimeric structure can allow the lectin domains to come within close proximity of each other and cooperate in binding IgE with high affinity (FIG. 10).

In order to develop effective therapeutic measures to restore airway homeostasis, it is vital to discover a novel pathway that the airway epithelium efficiently transports the applied immunotherapeutic protein. CD23-mediated transfer, if functional, might allow the patients to specifically utilize IgE Fc-fused immunotherapeutic proteins to regulate and dampen the intensity of allergic inflammation in airway. CD23 may offer a distinct advantage over current asthma therapy in that it can allow proteins to be efficiently delivered into the airway by avoiding the immunosuppressive effects from a systemic administration. More importantly, additional benefit of delivering IgE Fc-fusion protein over IgG Fc-fusion protein for regulating the allergic inflammation is that IgE Fc has a short half-life (2.5 days). The half-life of IgG Fc is about 23 days. This may avoid broad immunosuppressive effects in patients by using an immunosuppressive protein. Further, IgE Fc moiety in fusion proteins can have an inhibitory activity for IgE Fc-facilitated binding of allergen-IgE complexes to B cells or IgE-dependent Th2 cytokine release from mast cells and basophils. Therefore, this study provides support that CD23 acts as a trafficking receptor to ferry IgE Fc-fused immunotherapeutic proteins across the airway epithelial barrier in the context of allergic inflammation

IgE-mediated allergic inflammation occurs when allergens cross-link antigen-specific IgE's on the surface of immune cells, thereby triggering the release of inflammatory mediators as well as enhancing antigen presentations. IgE is frequently present in airway secretions; its level can be enhanced in human patients with allergic rhinitis and bronchial asthma. However, it remains unknown how IgE appears in airway secretions. CD23 is constitutively expressed in established or primary human airway epithelial cells; its expression was significantly up-regulated when airway epithelial cells were subjected to IL-4 stimulation. In a transcytosis assay, human IgE were transported across the polarized human airway epithelial cell line Calu-3 (FIG. 11). Exposure of Calu-3 monolayer to IL-4 stimulation also enhanced the transcytosis of human IgE. In addition, CD23 specific antibodies or soluble CD23 significantly reduced the efficiency of IgE transcytosis, indicating a specific receptor-mediated transport by CD23. Transport of IgE was further verified in primary human bronchial epithelial cells. Human CD23 molecule is capable of mediating IgE transcytosis across polarized human airway epithelial cells.

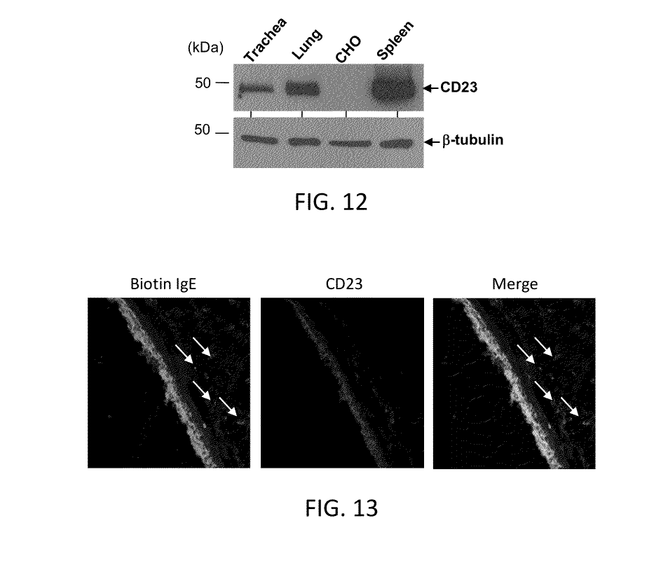

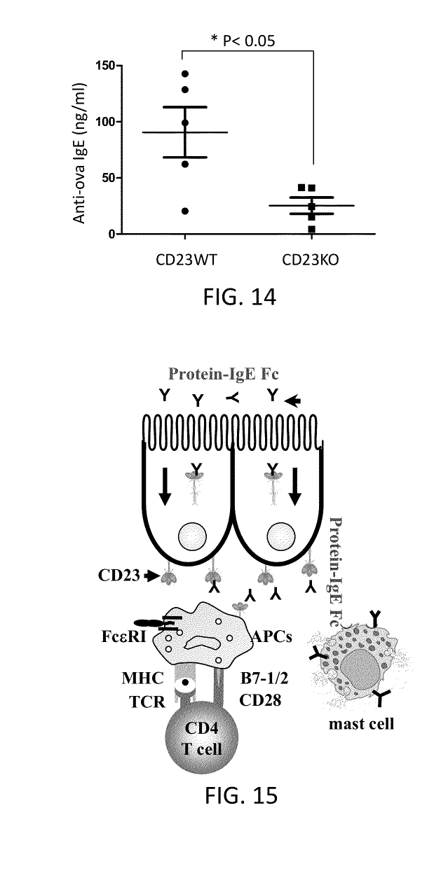

Mouse CD23 expression in the airway epithelial cells has also been characterized. Frozen section of mouse lung or trachea tissue was stained by immunohistochemistry with mouse CD23 specific B3B4 mAb. All staining reactions were accompanied by an IgG2a negative control. The results showed that mouse CD23 was expressed in airway epithelial cells. To verify this, airway epithelial cells were isolated for examining CD23 expression according to the procedures previously described by others. The cell lysates were subjected to Western blot analysis. Similar to human CD23, mouse CD23 was expressed in the airway epithelial cells of both trachea and lung (FIG. 12). To further illustrate CD23 captures IgE in the airway tract, biotin-labeled IgE (20-50 μg) were intranasally (i.n.) inoculated into wild type mice. By confocal microscope, intranasally inoculated biotin-labeled IgE (left panel) and CD23 (middle panel) in the trachea were well colocalized, indicated in merged picture (FIG. 13). Some green particles appeared in the laminar propria (arrow), which may represent the transported IgE molecules. To further show the IgE transport across the airway barrier in vivo, biotin-labeled ovalbumin specific IgE was i.n. inoculated into WT and CD23 KO mice (FIG. 14). The bare detection of biotin-IgE in CD23 KO mice also ensures that the airway epithelial cells kept intact without leakiness. Nevertheless, rodent CD23 receptor can specifically transport IgE across airway mucosal barrier.

Observations on IgE transport within airway mucosal epithelia by CD23 indicate that CD23 can transport an immunotherapeutic protein, if fused with the IgE Fc, across the mucosal barrier. Several lines of evidence indicate that this pathway for direct shuttling of IgE Fc-fusion protein may be feasible. For example, in the rodent, IgE can be transported from the gut lumen, and an IgG Fc-fused vaccine protein can be transported across the lung mucosal barrier. Hence, if a protein is fused to the IgE Fc part, CD23 can allow the host to specifically sample this Fc-fused protein in mucosal lumen, followed by transport across the airway epithelial barrier (FIG. 15).

Th2-mediated inflammation is a key feature of asthma; many immunotherapeutic strategies are based upon counter-balancing the Th2 cytokines with blockers promoting Th1 responses, interrupting mast cell signaling, and blocking IgE mediated pathways. These are likely to be due, at least in part, to an allergen-blocking effect at the mast cell level and/or at the level of the antigen-presenting cell that prevents IgE-facilitated activation of T cells.

2. Expression of IgE Fc-Fused Proteins and Transport of Fusion Protein In Vitro and In Vivo.

CD4+ T cell activation leading to cytokine production requires two signals from antigen presenting cells (APC): peptide-MHC I complexes and co-stimulation signal. B7 interaction with CD28 on T cells provides a co-stimulatory signals inducing T cells to proliferate (FIG. 15). Failure to receive this signal can result in anergy. CTLA-4 has a much higher affinity for B7 than does CD28 (FIG. 15). Consequently, CTLA-4 passes on a negative regulatory signal to CD4 T cells. Hence, the CD28-B7 pathway is important to allergen-induced inflammation in asthma.

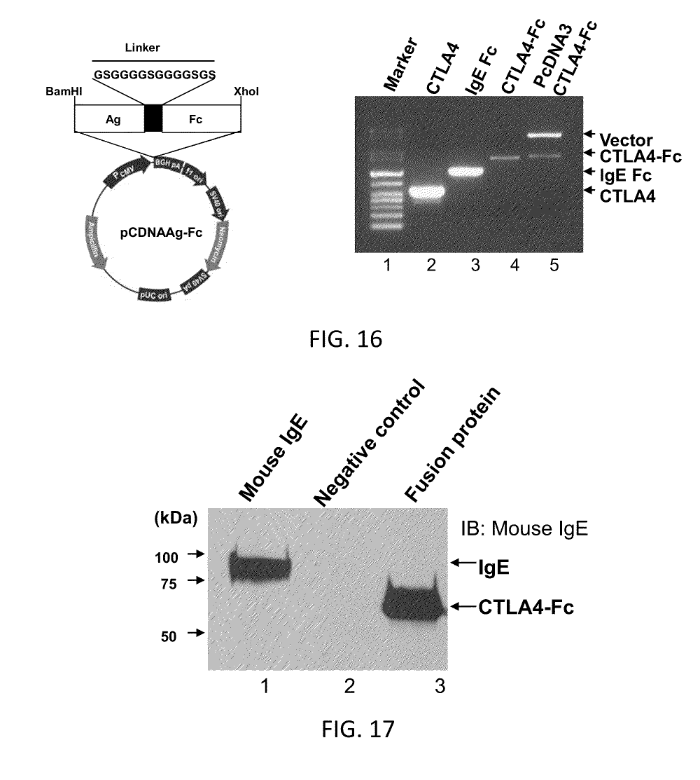

The Fc fragment of mouse IgE has been amplified from SPE-7 IgE hybridoma. The amplified IgE Fc cDNA was fused with the C-terminus of mouse CTLA-4 incorporating a short glycine linker (FIG. 16). To generate the appropriate fusions, PCR was used to amplify the extracellular domains of CTLA-4 and IgE Fc part, respectively. These amplifications were done with primers that contain the restriction sites incorporated for use in cloning into the pcDNA3. In designing the PCR, codons for glycine and serine residues were added upstream of the codons encoding the mouse IgE Fc fragment by synthesizing of the appropriate oligonucleotides. Fusions were then performed in PCR-based gene assembly approach by mixing the cDNA for CTLA-4 and the Fc fragment. Each construct was verified for fidelity of amplification and cloning by DNA sequencing. The plasmid containing the chimeric CTLA4-Fc fragment was transfected into Chinese hamster ovary (CHO) cells. Western blot using antibodies specific for murine IgE Fc and CTLA-4 was performed to assess the recombinant fusion proteins. The highest secreting clones were screened under G418 selection in serum-free medium (FIG. 17). A functional test of IgE Fc-domain can be verified in vitro by precipitation with CD23. This will allow for the determination of whether the Fc portion of IgE in the fusion proteins maintain all structures necessary for binding to CD23. The biological activity of CTLA-4 in fusion proteins can be measured by inhibitory activity on mixed lymphocyte culture or analysis for CTLA-4 and B7 interaction as previously described. A T-Gel affinity column, which binds to the IgE Fc portion in fusion proteins, can be used to purify CTLA4-Fc proteins.

To test the theory that CD23 functions as a receptor for the transport of therapeutic protein across the airway epithelial barrier, the following experiments can be performed. First, the transport of CTLA4-Fc proteins can be tested in polarized mouse airway epithelial cells in a transwell model as recently described for IgE transport; this can verify whether CTLA4-Fc maintains structural integrity and biological function. Second, in vivo transport of the fusion proteins can be conducted. Biotin-labeled CTLA4-Fc or commercial CTLA-4 (20 μg/30 μl) can be i.n. administered to assess their ability across the airway in vivo. This can show whether the levels of CD23 are sufficient to transport CTLA4-Fc and identify a CTLA4-directed immune regulation. The transported biotin-labeled CTLA4-Fc or biotin-CTLA4 in bloodstream can be measured by ELISA at 6-10 hr following the inoculation. The success in transport can also remove the concern that the CTLA4-Fc is of insufficient avidity, or that the CD23 levels are too low to allow efficient delivery. The correlation between CD23 and Fc-dependent absorption can also be confirmed using CD23 KO or transgenic mice. By using CD23 KO or transgenic mice, the amount of CTLA4-Fc or CTLA-4 in the bloodstream can be consistent with the idea that absorption of CTLA4-Fc across the airway epithelia depends on CD23 expression.

To avoid CTLA4-Fc fusion proteins misfolding, a minimal hydrophobicity and charge of the polyglycine linker can be used to maximize the opportunity for the proteins to fold into their native conformations. The successful production of CTLA4-IgG Fc indicates that this might not be a concern. The affinity of the CTLA4-Fc with soluble CD23 in comparison with the affinity of IgE can be analyzed by a BIACORE assay, if necessary. This would allow direct measuring of the fusion protein's ability to bind to CD23.

3. Characterization of the Efficacy to CTLA4-Fc Targeted to CD23 Pathway in Asthma Model.

The mouse model of airway inflammation has many features observed in humans. Previous studies have reported that suppression of the Th2 immune response correlates with local administration of CTLA4-1 g (IgG). More importantly, there is no documentation that CD23 directly mediates a delivery for any immune therapeutic proteins. Hence, CTLA4-Fc can be applied as a model fusion protein that is capable of binding CD23 to define the consequences of CTLA4-Fc entry via CD23-mediated transport across the airway mucosal barrier. Therapeutic effects in initiation of allergic responses relevant to this specific transport can be studied. The asthmatic condition can be initiated and progressed at the level of CD23-mediated transport of the CTLA4-Fc across airway epithelium. An OVA asthma model can be used to test the functions of CD23-mediated IgE transport. Specifically, CTLA4-Fc can be administered to mice before sensitization or before or during local lung challenge with OVA. In vivo analysis of airway hyperresponsiveness (AHR) to methacholine, eosinophilic inflammation, elevations in serum IgG1 and IgE levels, and levels of Th2 cytokines in lung can be performed.

The physiological experiments to show the specificity of CTLA4-Fc transport to attenuate the allergic inflammation can comprise of four groups: CTLA4-Fc/OVA, CTLA-4/OVA, OVA, and PBS. It should be noted that an additional control using commercially available CTLA-4 unlinked to an Fc fragment can not only show the transport efficiency of CTLA4-Fc by CD23 in vivo, but also help determine the magnitude of any observed attenuation in allergic inflammation. Groups of six-week-old Balb/c mice can be sensitized by i.p. injection of OVA and challenged with nebulized OVA. Four days before the inhalation challenge, the sensitized mice can be subjected to intranasal delivery of 25 μg/30 μl CTLA4-Fc, CTLA-4 proteins, or 30 μl PBS. Alternatively, CTLA4-Fc can also be i.n. applied before sensitization or after the challenge.

Use of CD23 KO mice can be potentially problematic given the expression of CD23 on multiple cell types. To definitively determine the role of CD23 on the airway epithelium in transporting CTLA4-Fc, the CD23 chimeras can be used. 3-5-week old CD23-deficient or WT recipient mice can be sublethally irradiated with 900 rad. On the same day, 5×10⁴ bone marrow cells can be collected from WT mice bone marrow donors and infuse via the tail vein. Control WT/WT chimeras can be created in parallel to KO/WT chimeras. Blood can be collected in EDTA-containing tubes at regular intervals, and the hematological parameters can be determined with a Technikon H1E analyzer. These mice can be kept in isolators and neomycin containing water is provided for 10-12 weeks. After 7-8 week of engraftment, hemopoietic reconstitution can be confirmed by analyzing different leukocyte populations in spleens. To permit complete chimeras in the lung, 10-12 weeks of reconstitution time can be allowed before we start experiments; the degree of chimeras of CD19+MHCII+ B cells, CD11c+MHCII+ DCs and alveolar macrophages, known to be slowly repopulated after irradiation, can be confirmed by measuring CD23 positivity after 12 weeks of chimeras and by immunostaining CD23 on cryo-sections of lungs and trachea, followed by confocal imaging. Splenic CD19*B cells are used as the control for CD23 chimeras.

To determine whether administrations of CTLA4-Fc blocks or attenuates the T cell mediated immune response, several important criteria can be used to measure the success. Most importantly, it should be emphasized that all of the protocols necessary to analyze inflammatory parameters have been well-established in our lab and our collaborators' lab. Airway histopathology can be observed along with analysis of other immunological assays.

For OVA immunization and challenge, mice can be i.p. immunized with 10 μg of OVA in 2 mg aluminum hydroxide. After 2 weeks, the mice can be given nebulized OVA for 30 min every day for 3-6 days. Control mice can be exposed to the nebulized PBS, pH 7.2. The BAL fluid can be collected after various time intervals following airway OVA challenge. The fluid can be evaluated for its IgE levels.

The treated mice can be tested for responses to asthma at various times after challenge. IL-4 level in the BAL can be measured. The cells in the BAL can be analyzed by staining with CD11b, F4/80, CCR3, and Siglec F Abs by FACS for the presence of Mφ and eosinophils and by differential staining after cytospin. After lavage, lungs can be fixed in formalin; histological sections can be prepared in paraffin. Sections can be stained using H&E or periodic acid-Shiff (PAS). These sections can be examined by veterinary pathologists without prior knowledge of the treatment groups for the extent of lung inflammation, goblet cell metaplasia, mucous production, and smooth muscle cell hyperplasia using morphometric techniques. The degree of the effect can be ranked 0-3+. In some experiments, single cell suspensions can be prepared from perfused lung tissue and the draining lymph nodes and analyzed by FACS for CD11b, F4/80, SiglecF, CCR3. In separate groups of mice, airway hyperreactivity to a challenge of methacholine can be analyzed using a Flexivent apparatus.

To examine the effects of CTLA4-FC treatment just before or after local lung challenge. PBS- and OVA sensitized mice can be i.n. or intratracheally given 25 μg CTLA4-Fc or CTLA4 control. The effect of CTLA4-Fc on OVA-induced pulmonary inflammation can be assessed by examining the cellular composition in BAL fluids. If the treatment is functional, OVA-induced pulmonary eosinophilia can be virtually ablated by treatment with CTLA4-Fc. To determine whether CTLA4-Fc treatment affects lung cytokine levels, mRNA levels IL-4, IL-5, IL-10, and IFN-γ can be measured in whole lungs from PBS- and OVA-sensitized and -challenged animals by RT-PCR. Also, IL-4, IL-5, IL-13 and IFN-γ can be measured in the BAL by ELISA. These experiments are designed to determine whether CD23 dependent CTLA4-Fc transport can preferentially ablate the production of Th2 cytokines. To show the total IgE levels in OVA-exposed, CTLA4-Fc-treated and control animals, IgE can be measured in the BAL by ELISA following administration of CTLA4-Fc either before sensitization or before challenge. This test can be a more sensitive assay to measure the IL-4 levels because higher doses and more prolonged stimulation of IL-4 are necessary for B cells to switch from IgG to IgE.

Untreated OVA-sensitized and challenged mice can show higher levels of IL-4 and more pronounced and rapid eosinophilia in BAL fluid than those receiving OVA alone. In contrast, control mice sensitized to OVA but exposed to aerosolized PBS during challenge do not show detectable levels of eosinophils. The levels of IgE in general can be correlated with eosinophilia in BAL. Airway delivery of CTLA4-Fc by CD23 can restore a balanced T cell immune response and attenuate airway inflammation. The blockade of B7/CD28 interactions by CTLA4-FC, either before local OVA challenge or during the time of challenge, can effectively abrogate the development of OVA-induced AHR to methacholine and eosinophilic inflammation. Such a decrease can exhibit a dose-dependent manner of CTLA4-FC treatment. The marked eosinophilic and lymphocyte infiltration in the BAL can be suppressed to a significant degree by CTLA4-Fc treatment, resulting in a reduction of peribronchial inflammation scores and serum IgE levels in OVA-sensitized and challenged mice during the effector phase. All these results can determine whether or not CD23-mediated CTLA4-FC transport can be therapeutically effective for the modulation of inflammation. The CD23 chimeric mouse can allow for direct and specific determination of the transport function of CD23 on the airway epithelium in the delivery of CTLA4-Fc fusion proteins. Data from these experiments can show that CD23-mediated delivery of therapeutic proteins effectively block the airway allergic inflammation.

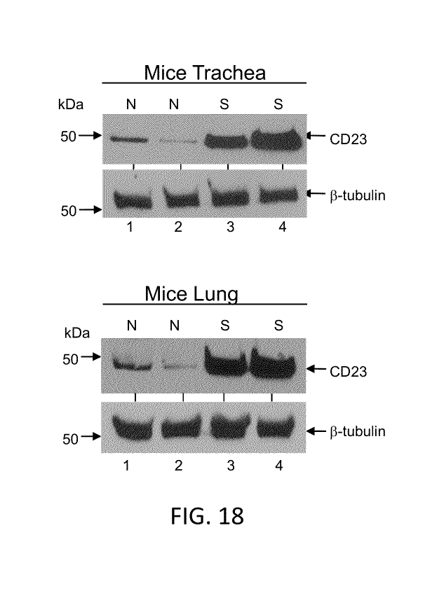

In some instances, an irrelevant IgE Fc fusion protein can be used as a negative control to show that the effects are truly due to the CTLA4 portion in fusion proteins, not simply as a nonspecific response to CD23 triggering upon Fc binding. Furthermore, amino acid residues lysine 352 located at the interface between CH2 and CH3 domains of human IgE have been identified by mutagenesis to be of particularly functional significance to this binding. Mapping a homologous amino acid in mouse IgE Fc which is responsible for binding to CD23 can be performed. A fusion protein CTLA4-Fc/mut which is unable to bind to CD23 can be generated and used as a negative control for delivery. Second, given CD23 expression on a wide variety of cells together with the reported increases in its sheddase ADAM10 during an inflammatory response, the utility of CD23 to transport immunotherapeutic for the treatment of asthma may be uncertain. However, data has shown that the majority of CD23 was located inside airway epithelial cells. Although the expression of sheddase ADAM10 is enhanced during inflammation, the level of CD23 expression was also concomitantly increased (FIG. 18). This remarkable increase for CD23 expression in tracheal or lung epithelial cells can enhance the efficiency of CD23 delivery of CTLA4-Fc. Transport of IgE was significantly enhanced when human Calu-3 cells were exposed to IL-4. Nevertheless, airway epithelial cells over-expressing ADAM10 protein can be used to transport CTLA4-Fc protein in order to exclude this possibility. Third, DCs present intercalating along the airway epithelium also express the high affinity IgE receptor, FcεRI. Therefore, uptake of the CTLA4-Fc fusion proteins by DCs could possibly confound the results of a CD23-specific effect. CD23 KO and chimeras can address this concern. Also, a recent study indicates that the uptake of IgG Fc fusion proteins by airway DCs is least possible. Fourth, development of OVA-based allergic model requires repetitively priming, this can affect the structural integrity of airway epithelial cells and cause the leakiness. To avoid this, we can intratracheally expose DO11.10 mice which have an expanded OVA TCR repertoire, to limited aerosols of OVA before or after administration of CTLA4-Fc proteins. The therapeutic effect can be fully analyzed.

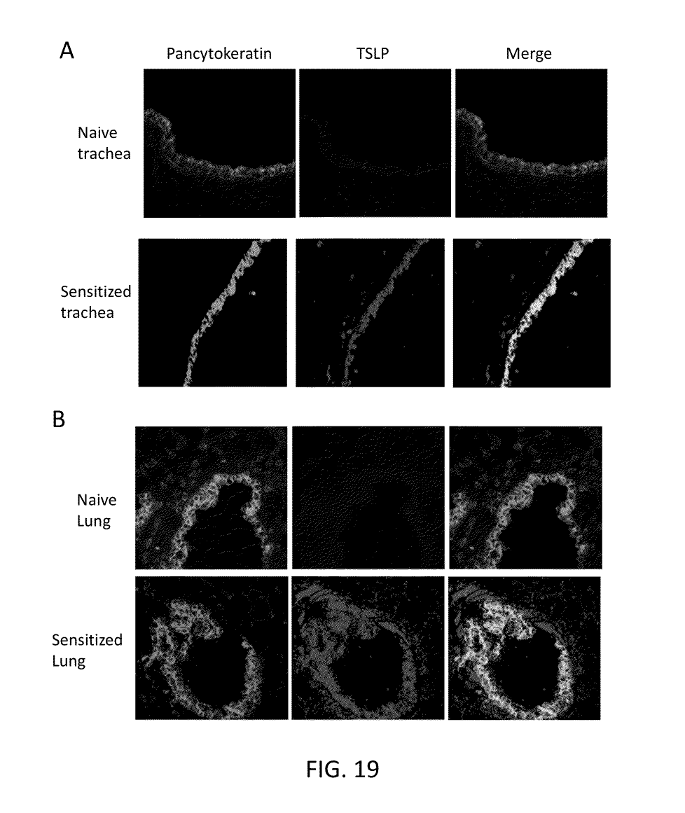

The impact of exposure to the CTLA4-Fc on a more clinically relevant allergen house dust mite or ragweed-induced allergic airway inflammation and its consequences for tissue remodeling and lung physiology in mice can be investigated. A variety of immunotherapeutic strategies have been reported for modulating Th2 immune responses to allergens leading to protection from allergic airways disease. Among these, IL-10, TGF-β, activin-A, thymic stromal lymphopoietin (TSLP) antagonist, etc., have been proposed. One study showed TSLP was largely produced by trachea (panel A) or lung (panel B) epithelial cells in OVA sensitized mice (FIG. 19). The blocking TSLP signaling with soluble TSLP receptor (TSLPR) on asthma development is effective. Studies to identify the CD23-mediated delivery of these therapeutic proteins (IL10-Fc, TSLPRFc) are being investigated.

Due to potential variability of immunological assays and animal experiments, all experiments can be repeated at least three times. Statistical analysis can be performed by Student's t-test. Statistical significance at a P value of less than 0.05 can be determined by ANOVA using Statview 4.5 statistical analysis software. The Bonferroni procedure can be used for multiple comparisons between the means of groups.

We claim:

1. A composition comprising an Fc portion of IgE coupled to an agent.

2. The composition of claim 1, wherein the agent is an antigen.

3. The composition of claim 2, wherein the antigen is an allergen.

4. The composition of claim 2, wherein the antigen is derived from a virus, bacteria, parasite or fungus.

5. The composition of claim 3, wherein the allergen is an antigen derived from pollens, dust, mites, molds, spores, dander, insects or foods.

6. The composition of claim 5, wherein the allergen is urushiols (pentadecylcatechol or heptadecylcatechol) or sesquiterpenoid lactones.

7. The composition of claim 2, wherein the antigen is a tumor antigen.

8. The composition of claim 2, wherein the antigen is a cell surface, cytoplasmic, nuclear, or mitochondrial antigen.

9. The composition of claim 1, wherein the agent is an immunotherapeutic.

10. The composition of claim 9, wherein the immunotherapeutic is selected from the group consisting of Cytotoxic T lymphocyte antigen 4 (CTLA4), Soluble IL-4 receptor, Soluble IL-13 receptor, IL-5R, Thymic stromal derived lymphopoietin receptor (TSLPR), IL10, IL-9 receptors, IL-17 receptors, IL 21 receptors, IL-25 receptors, IL-31 receptors, IL-33 receptors, transforming growth factor beta (TGFbeta), transforming growth factor beta receptors, Histamine receptors, Prostaglandin receptors, FcepsilonRI alpha, programmed death 1 (PD1), Flt3-ligand, leukotriene receptor, Tumor necrosis factor (TNFR), LIGHT receptor, OX40L, IL-1 beta receptor, c-kit, ADAM, soluble intercellular adhesion molecule 1 (sICAM-1), soluble IL-2R, CD48, Pulmonary surfactant protein D (SPD), soluble β2-Adrenergic receptor, B7-1, B7-2, B7-H1, B7-H2,leukocyte-associated immunoglobulin-like receptor 1 (LAIR1), lymphocyte activation gene 3 protein (LAG3), CD160, LAG3, lymphocyte activation gene 3 protein (TIGIT), type I transmembrane (or T cell) immunoglobulin and mucin (TIM3), B and T lymphocyte attenuator (BTLA).

11. The composition of claim 1 further comprising a pharmaceutically acceptable carrier.

12. A vaccine comprising the composition of claim 2.

13. A method of treating airway inflammation comprising administering to a subject an effective amount of the composition of claim 9.

14. A method of interfering with Th2 cell activation comprising administering to a subject an effective amount of the compositions of claim 9.

15. A method of blocking mast cell degranulation comprising administering to a subject an effective amount of the composition of claim 1.

16. A method of decreasing proinflammatory cytokines in the bronchoalveolar lavage fluid (BALF) comprising administering to a subject an effective amount of the composition of claim 9.

17. The method of claim 13, wherein the administration is intranasal.

18. A method of modulating the immune system comprising administering to a subject an effective amount of the composition of claim 1.

19. A method of delivering an antigen across airway epithelial cells comprising administering to a subject an effect amount of the composition of claim 2.

20. A method of delivering an immunotherapeutic across airway epithelial cells comprising administering to a subject an effect amount of the composition of claim 9.
